(19) United States
(12) Patent Application Publication
(10) Pub. No.: US 2013/0172895 A1
(43) Pub. Date: Jul. 4, 2013

(54) DEVICES, SYSTEMS AND METHODS FOR TISSUE MODIFICATION

(71) Applicants: Michael P. WALLACE, Pleasanton, CA (US); Roy LEGUIDLEGUID, Union City, CA (US); Benjamin Kao-Shing SUN, San Francisco, CA (US); Christopher BAGLEY, Santa Clara, CA (US); Robert GARABEDIAN, Sunnyvale, CA (US); Bryan KNODEL, Flagstaff, AZ (US); Brian S. BOWMAN, Carlsbad, CA (US)

(72) Inventors: Michael P. WALLACE, Pleasanton, CA (US); Roy LEGUIDLEGUID, Union City, CA (US); Benjamin Kao-Shing SUN, San Francisco, CA (US); Christopher BAGLEY, Santa Clara, CA (US); Robert GARABEDIAN, Sunnyvale, CA (US); Bryan KNODEL, Flagstaff, AZ (US); Brian S. BOWMAN, Carlsbad, CA (US)

(21) Appl. No.: 13/728,767

(22) Filed: Dec. 27, 2012

Related U.S. Application Data
(60) Provisional application No. 61/581,589, filed on Dec. 29, 2011.
(60) Provisional application No. 61/666,427, filed on Jun. 29, 2012.

(51) Int. Cl. A61B 17/16

(52) U.S. Cl. CPC A61B 17/1604
USPC 606/83

(57) ABSTRACT

Devices and methods of modifying tissue for low profile and ultra profile rongeur devices to treat spinal tissue. These devices may include a curved or curveable distal region; the cutting member may be configured to operate in the curved region. Also described herein are tissue modification devices that may be flexible or bendable for positioning in the tissue (including the spinal region) but can be made rigid once in position, or otherwise fixed in place to allow leverage when modifying the tissue.

### CROSS REFERENCE TO RELATED APPLICATIONS

This patent application claims priority to U.S. Provisional Patent Application No. 61/581,589, filed on Dec. 29, 2011, titled “SYSTEMS AND METHODS FOR SPINAL MODIFICATION,” which is herein incorporated by reference in its entirety.

This patent application also claims priority to U.S. Provisional Patent Application No. 61/666,427, filed on Jun. 29, 2012, titled “TISSUE MODIFICATION DEVICES,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are systems and methods for tissue cutting and removal, including medical/surgical devices and methods. For example, described herein are surgical systems, including powered surgical files, for cutting, removing, grinding, shaping and sculpturing bone and/or tissue material.

More specifically, the devices described herein relate to tissue modification devices that may be thin (including flat), curved and/or curveable and include one or more active cutting members, such as a closable jaw. Also described are methods of modifying tissue using such devices, particularly for treatment of spinal stenosis.

### BACKGROUND

A significant number of surgical procedures involve modifying tissue in a patient's body, such as by removing, cutting, shaving, abrading, shrinking, ablating or otherwise modifying tissue. Minimally invasive (or “less invasive”) surgical procedures often involve modifying tissue through one or more small incisions or percutaneous access, and thus may be more technically challenging procedures. Some of the challenges of minimally invasive tissue modification procedures include working in a smaller operating field, working with smaller devices, and trying to operate with reduced or even no direct visualization of the tissue (or tissues) being modified. For example, using arthroscopic surgical techniques for repairing joints such as the knee or the shoulder, it may be quite challenging to modify certain tissues to achieve a desired result, due to the required small size of arthroscopic instruments, the confined surgical space of the joint, lack of direct visualization of the surgical space, and the like. It may be particularly challenging in some surgical procedures, for example, to cut or contour bone or ligamentous tissue with currently available minimally invasive tools and techniques. For example, trying to shave a thin slice of bone off a curved bony surface, using a small-diameter tool in a confined space with little or no ability to see the surface being cut, as may be required in some procedures, may be incredibly challenging or even impossible using currently available devices.

One area of surgery which would likely benefit from the development of less invasive techniques is the treatment of spinal stenosis. Spinal stenosis occurs when nerve tissue and/or the blood vessels supplying nerve tissue in the spine become impinged by one or more structures pressing against them, causing symptoms. The most common form of spinal stenosis occurs in the lower (or lumbar) spine and can cause severe pain, numbness and/or loss of function in the lower back and/or one or both lower limb.

FIG. 1 is a top view of a vertebra with the cauda equina (the bundle of nerves that extends from the base of the spinal cord) shown in cross section and two nerve roots branching from the cauda equina to exit the central spinal canal and extend through intervertebral foramina on either side of the vertebra. Spinal stenosis can occur when the spinal cord, cauda equina and/or nerve root(s) are impinged by one or more tissues in the spine, such as buckled or thickened ligamentum flavum, hypertrophied facet joint (shown as superior articular processes in FIG. 1), osteophytes (or “bone spurs”) on vertebrae, spondylolisthesis (sliding of one vertebra relative to an adjacent vertebra), facet joint synovial cysts, and/or collapse, bulging or herniation of an intervertebral disc. Impingement of neural and/or neurovascular tissue in the spine by one or more of these tissues may cause pain, numbness and/or loss of strength or mobility in one or both of a patient's lower limbs and/or of the patient's back.

In the United States, spinal stenosis occurs with an incidence of between 4% and 6% (or more) of adults aged 50 and older and is the most frequent reason cited for back surgery in patients aged 60 and older. Patients suffering from spinal stenosis are typically first treated with conservative approaches such as exercise therapy, analgesics, anti-inflammatory medications, and epidural steroid injections. When these conservative treatment options fail and symptoms are severe, as is frequently the case, surgery may be required to remove impinging tissue and decompress the impinged nerve tissue.

Lumbar spinal stenosis surgery involves first making an incision in the back and stripping muscles and supporting structures away from the spine to expose the posterior aspect of the vertebral column. Thickened ligamentum flavum is then exposed by complete or partial removal of the bony arch (lamina) covering the back of the spinal canal (laminectomy or laminotomy). In addition, the surgery often includes partial or complete facetectomy (removal of all or part of one or more facet joints), to remove impinging ligamentum flavum or bone tissue. Spinal stenosis surgery is performed under general anesthesia, and patients are usually admitted to the hospital for five to seven days after surgery, with full recovery from surgery requiring between six weeks and three months. Many patients need extended therapy at a rehabilitation facility to regain enough mobility to live independently.

Removal of vertebral bone, as occurs in laminectomy and facetectomy, often leaves the affected area of the spine very unstable, leading to a need for an additional highly invasive fusion procedure that puts extra demands on the patient's vertebrae and limits the patient's ability to move. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Thus, while laminectomy, facetectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients.

Therefore, it would be desirable to have less invasive methods and devices for modifying target tissue in a spine to help ameliorate or treat spinal stenosis, while inhibiting unwanted damage to non-target tissues. Ideally, such techniques and devices would reduce neural and/or neurovascular impingement without removing significant amounts of vertebral bone, joint, or other spinal support structures, thereby avoiding the need for spinal fusion and, ideally, reducing the long-term morbidity resulting from currently available surgical treatments. It may also be advantageous to have minimally invasive or less invasive tissue modification devices capable of treating target tissues in parts of the body other than the spine. At least some of these objectives will be met by the present invention.

As mentioned, it would be desirable to provide treatment devices and methods for treating a patient that permit tissue to be removed to enlarge the space for nerves without weakening the back or afflicted joint. Further, it would be helpful to provide devices suitable for operating in the already narrowed and constricted confines of the patient's back (e.g., neural foramen) while providing sufficient leverage to allow efficient cutting of the tissue. Thus, described herein are devices and methods for treating tissue that may address some of these issues.

U.S. patent application Ser. No. 11/406,486 (issued as U.S. Pat. No. 7,938,830) and U.S. patent application Ser. No. 13/078,376 (publication number US 2011/0190772) describe powered mechanical tissue modification devices, each of which is herein incorporated by reference in its entirety. The devices and methods described herein improve upon the methods and devices described in these cases.

In general the devices and systems described herein may be used to remove tissue, including bony and/or difficult to access tissues, in a manner that is not possible as effectively with prior art devices.

### SUMMARY

In general, described herein are tissue modification devices, including rongeur devices. A rongeur device is a surgical instrument that may include a tip for removing (e.g., “biting” or gouging out bone). These devices may be unimanual, meaning that they can be operated to cut tissue using a single hand, and may be stiff or stiffenable. In some variations the devices describe herein are low-profile or ultra low-profile, so that the cutting portion of the device may fit within even narrow body region, including a spinal foramen. In some variations the devices described herein are curved or bent at their distal end; the cutting element may traverse or span this curve or bend, allowing the device to cut, typically from a lateral window or region of the device. In some variations the device is configured to be bent or curved while inserting, yet be rigid or stiff prior to actuating the device, allowing sufficient leverage to cut the tissue.

For example, described herein are ultra low-profile rongeur device for cutting a target tissue, the device comprising: an elongate body having a distal portion having a height and width, wherein the distal portion of the device is configured to be passed into an epidural space and has a height that is less than about 3 mm; a first blade movably disposed across the width of one side of the distal portion of the elongate body configured to cut target tissue; and a handle at the proximal end of the body, wherein the handle includes an actuator configured to drive the first blade towards a second blade to cut target tissue.

The distal portion of the elongate body may be bent or curved, and first blade may be configured to move along the curved distal portion of the elongate body. In some variations the actuator is configured to pull the second blade toward the first blade. Alternatively, the actuator may be configured to push the first blade toward the second blade. I general, the device may be configured to cut target tissues within the lateral recess of a spine. For example, the width may be significantly greater than the height of the distal portion of the elongate body. The distal portion may have a width that is greater than about 4 mm.

The device may also include a rigid shaft region between the distal portion of the elongate body and the proximal handle. In some variations this shaft is flexible or bendable, but may be rigidified or stiffened prior to actuating.

The portion of the device may be curved such that there is an angle between the rigid shaft and the distal portion of the elongate body. The angle may be between 180 degrees and 90 degrees. In some variations the angle may be less than 90 degrees.

In some variations, the device includes an opening through the first or second blade through which cut tissue may pass.

The device may also include one or more flexible tendons coupled to the first blade and the actuator and configured to move the first blade relative to the second blade. A tendon is typically an elongate member and have sufficient column strength to push the first blade relative to the second blade. The tendon may be a wire, ribbon, etc. and may have a round, triangular, square, oval, rectangular, or other cross-sectional profile. The one or more flexible tendons may comprise a plurality of adjacently arranged wires. For example, a tendon may be a shape memory alloy, such as Nitinol.

For example, described herein are ultra low-profile rongeur devices for cutting a target tissue, comprising: an elongate body comprising a distal portion having a height and width and an elongate rigid shaft portion, wherein the distal portion of the device has a curve relative to shaft, further wherein the distal portion is configured to be passed into an epidural space and has a height that is less than about 3 mm; a first blade that is movably disposed across the width of one side of the distal portion of the elongate body configured to cut target tissue; and one or more flexible tendons coupled to the first blade and configured to drive the first blade along the curve of the distal portion and against a second blade in the distal end region to cut target tissue. The device may also comprise a handle at the proximal end of the body, wherein the handle includes an actuator configured to move the one or more flexible tendons.

The distal portion may have a width that is greater than about 4 mm. As mentioned, the device may have an opening through the first or second blade through which cut tissue may pass.

### BRIEF DESCRIPTION OF THE DRAWINGS

FIG. 1 is a top view of a vertebra with the cauda equina shown in cross section and two nerve roots branching from the cauda equina to exit the central spinal canal and extend through intervertebral foramina on either side of the vertebra.

FIG. 2 is a top view of a vertebra shown in cross section with a conventional rongeur device.

FIG. 3 is a top view of a vertebra shown in cross section showing the central canal, lateral recess, and foraminal regions.

FIGS. 4-5B illustrate a particular example of target tissue that may be removed with the devices and methods described herein.

FIG. 6A shows a first tissue modification device compared to conventional (rigid) rongeur device; in this variation the tissue modification device includes a biting or cutting region that is low-profile, particularly as compared to prior art devices.

FIG. 6B shows a side view of another variation of a tissue modification device compared directly to a prior art rongeur device; the current device is in front of the prior art rongeur device.

FIG. 6C shows another comparison of one variation of a tip region compared to the prior art rongeur device showing representative thicknesses. Thus the device illustrated is a low-profile (e.g., less than about 5 mm high, less than about 4 mm high, less than about 3 mm high, less than about 2 mm high, etc.) or very low profile (less than about 3 mm high, less than about 2 mm high, etc.) rongeur device. Despite its low profile, the device may have a strength and stiffness/flexibility that is comparable or greater than that of the prior art rongeur.

FIGS. 7 and 8A-8B show another variation of a tissue modification device configured as a low-profile or extremely low profile rongeur-like device having a distal end that is curved and an upwardly-facing (e.g., towards the direction of the curve) cutting/biting mouth.

FIGS. 9A-9B illustrate operation of another variation of tissue-modification device configured as a low-profile rongeur device.

FIGS. 10A-10B illustrate operation of the distal cutting region including a biting jaw that slides to open and close, despite the curved proximal region. In some variations the biting region (“mouth”) facing upwards in the direction of the curvature, may also be curved.

FIGS. 11A-17B illustrate exemplary curved tissue modification devices for removing impinging tissue.

FIGS. 11A-11B show a variation of a curved tissue modification device including a distal biting/cutting region that faces into the curve. In this variation the jaw of the biting/cutting region slides and opens along the curvature as shown.

FIGS. 12A-12B illustrate another variation, similar to that shown in FIGS. 11A-11B, of a curved tissue modification device.

FIGS. 13A-13B illustrate another variation of a cutting device similar to the devices shown in FIGS. 11A-11B and 12A-12B, but having a squared cutting region.

FIGS. 14A-14C illustrate another variation of a tissue modification device having a curved cutting region (biting region) for removing tissue.

FIG. 15 illustrates another variation of a tissue modification device having a curved biting region; in this example, the device has a proximal handle with a control (trigger) for controlling the biting action sliding the upper jaw along the curved biting region to close it against a hooked complementary biting jaw that is fixed relative to the sliding upper jaw in this example. The intermediate region is somewhat rigid, as is the distal end, though the upper jaw may slide to cut tissue within the cutting region.

FIGS. 16A-16B illustrate the operation of the biting region.

FIGS. 17A-17B show side views of the biting region of the low-profiled, curved, rongeur shown in FIG. 15.

FIGS. 18A-18B illustrate a supported embodiment and a non-supported embodiment, respectively, of a tissue modification device having a low-profile biting region that may be curved, so that the biting jaw element for cutting the tissue may travel along a curved path.

FIGS. 19A-32B illustrate various blade embodiments and blade combinations.

FIGS. 19A-19D illustrate variation of biting jaws (blades) that may be used with variations of the tissue modification devices as described herein.

FIGS. 20A-20C illustrate another variation of a blade combination that may form part of the tissue modification device. In this variation the two biting members engage with an angled surface. In any of the variations of devices described herein one or both of the members forming the closable jaws may be fixed while one is movable, or both may be configured to move together to close and/or separate.

FIGS. 21A-21C illustrate side, top and bottom perspective views of another variation of biting members (blades) that may be used to form the jaws of a tissue modification device. In this variation the biting members may have a different thickness, as shown.

FIGS. 22A-22C illustrate side, top and bottom perspective views of another variation of biting members (blades) that may be used to form the jaws of a tissue modification device. In this example, the two members (first and second members) may each include an interdigitating region so that he first and second biting members interdigitate when combined, as shown in FIG. 22A.

FIGS. 23A-23C illustrate side, top and bottom perspective views of another variation of biting members (blades) that may be used to form the jaws of a tissue modification device. In this example, the first and second members meet on faces each having a ramping region so that each biting member has a cutting edge and the cutting edges meet when the biting members are closed.

FIGS. 24A-24E illustrate another pair of biting members. In this variation, at least one of the biting members includes a passage through which tissue (e.g., cut tissue) may pass. Cut tissue may be stored by the device or released back into the body. In some variations the device may include a removal mechanism for removing the cut tissue, such as an aspiration channel, which may be connected to the opening.

FIG. 25 shows a pair of biting members (blades) mounted to a tissue modification device. In this variation one of the blade is fixed, while the other blade is coupled to a set of parallel pushing tendons configured to drive the blade closed/open relative to the other biting member.

FIGS. 26A-26D illustrate another pair of biting members. FIG. 26A shows a top perspective view of the two biting members engaged with each other, while FIG. 26B shows a side perspective view of the engaged biting members. The first biting member is shown in FIG. 26C and the second biting member is shown in FIG. 26D. The biting members include pointed cutting teeth (triangular shaped) which may interdigitate when closed.

FIG. 27 shows another pair of biting members (blades) mounted to a tissue modification device. The biting members are similar to those shown in FIG. 26A-D.

FIGS. 28A-28B show top perspective and side views, respectively, of another pair of biting members. In this variation the first biting member (FIG. 28C) is configured with a cutting edge formed by the acutely angled profile of the distal end, in which the distal face of the member angles down from the upper surface as the surface extends proximally. The second biting member (FIG. 28D) has a complementary profile, in which the cutting surface is formed by an upper distal-facing sloped surface, so that the two engage to form a flat outer surface when the cutting mouth is closed.

FIGS. 29A-29B show top perspective and side perspective views, respectively of the biting members of FIGS. 28A-28B mounted to a tissue modification device.

FIGS. 30A-30C illustrate side perspective, side and top perspective views, respectively, of another variation of a pair of cutting members in which the first and second members engage along a cutting edge between the two members. The two members may not engage surface-to-surface, but merely edge-to-edge, as illustrated in FIG. 30B. Thus, the two members may or may not be complimentary along a surface.

FIGS. 31A-31B illustrate top perspective and side views, respectively, or another variation of a pair of biting/cutting members, in which the first member and second member at least partially engage with each other so that a portion of one of the two fits into an opening in the other (e.g. one of the two is housed at least partially within the other). FIG. 31C shows the first member, which includes an opening or channel into which a projecting portion of the second cutting member (FIG. 31D) fits.

FIGS. 32A-32B show top perspective and side views, respectively, of the cutting members of FIG. 31A mounted to the distal end of a low-profile tissue modification (configured as a low-profile uni-manual rongeur) device.

FIGS. 33A-34D illustrate various embodiments of tissue modification devices including tissue management elements.

FIG. 33A-33CB shows variations of tissue modification devices including cutting members that have an opening through which cut tissue may pass. FIG. 33A shows the distal end of the tissue modification device including a (fixed) second cutting member that has an opening; the body of the distal end is configured with a ramping region to guide the cut tissue out of the distal end of the device. FIG. 33B shows the second cutting/biting member isolated from the distal end of the device. FIG. 33C is another variation of a cutting member that may be fixed to the distal end of the device and also include a channel with an opening that guides the tissue from the lateral side of the distal end of the device.

FIGS. 34A-34D illustrate variation of the device including a compartment for storing cutting tissue. In FIG. 34A the compartment include a bias element (shown as a spring in this example) that is compressed as the compartment or channel fills with cut material. FIG. 34B shows another example in which the compartment includes a compressible material that compresses as the compartment fills with cut material. In FIG. 34C one or more walls of the compartment are expandable. In FIG. 34D a channel though the device may be included to capture and/or remove the cut tissue.

FIGS. 35-37 illustrate embodiments of the tissue modification device having an expandable blade region.

FIG. 35 illustrates one variation of a cutting device in which the distal cutting members may rotate up (away from the longitudinal axis of the device) to form a larger cutting “mouth” that potentially allows a deeper cut into the tissue.

FIGS. 36A-36B illustrates another variation in which the depth of the cutting region may be modified (increased/decreased) by displacing the movable cutting element (the proximal or first cutting/biting member) axially away from the long axis of the distal end of the tissue modification device. In FIGS. 36A-36B, a strap or guide may be controlled to displace the second member as shown.

FIG. 37 shows another variation of a tissue modification device in which the depth of the cut may be modified by displacing the cutting element. In this example, the cutting element may ride up in a channel to permit a deeper cut to be made.

FIGS. 38A-38B illustrate a tissue modification device having an alternative tissue modification mechanism. In this example, the cutting members include rotating burr(s) that may be rotated in opposite directions (e.g., towards each other) to cut/bite tissue. The rotating burrs may be arranged longitudinally along the distal end (cutting region) of the device.

FIGS. 39-56 show variations of devices for use in removing tissue, and particularly tissue from a subject's back. Any of these variations may be incorporated with a cutting window (e.g., first and second biting/pinching cutting members) as illustrated above. Manual and/or powered cutting mechanisms may be included.

FIG. 39 shows a variation of tissue modification device including a bendable, lockable distal end. In this example, the angle of the distal end of the device may be modified as illustrated.

FIG. 40 illustrates another variation of a tissue modification device including a hinged or bendable and lockable distal end region. The distal end may also include a cutting region, including a cutting window as described above.

FIG. 41 illustrates another variation of a tissue modification device in which at least a portion of the device (proximal to the distal end, including an intermediate portion immediately adjacent to the distal end or cutting window region) is formed of lockable links that may be stiffened and/or locked into position.

FIG. 42 shows a flexible guide with a stiffenable/ridigifying member.

FIGS. 43A-43B illustrate a bendable device.

FIG. 44 illustrates one variation of a device having a region of flexible connecting wires.

FIG. 45 shows a variation of a device having a flexible blade with an infallible anchoring member.

FIG. 46 shows a device having a cutting surface that is hinged/extendable from the body of the device.

FIG. 47 shows a device including a distal end region having different distal, proximal, and middle compositions.

FIG. 48 is another variation of a device having regions of different composition and function.

FIG. 49 shows another variation of a device having a hinged region between the proximal and distal regions.

FIG. 50 illustrates another variation of a device having a rotational cutter at the distal end (e.g., configured as auger blades).

FIG. 51A shows a perspective view of another variation a tissue modification device. In FIG. 51A the rotating cutter (shown in greater detail in FIG. 51B) includes a blade that rotates (e.g., alternating clockwise and counterclockwise).

FIG. 52A shows another example of a tissue modification device including a rotating (e.g., ball) cutter/blade. FIG. 52B shows an enlarged view of the ball with a sharp-edged cutting cavity formed therein.

FIG. 53 shows another example of a tissue modification device having a continuously driven blade/cutting element.

FIG. 54 illustrates one variation of a device including a backing along which the bladed element may move over; the backing may be trackless.

FIG. 55 shows a variation of a tissue modification device in which the blades (cutting element(s)) are biased and may be actuated against the bias to move the blades reciprocally to cut tissue.

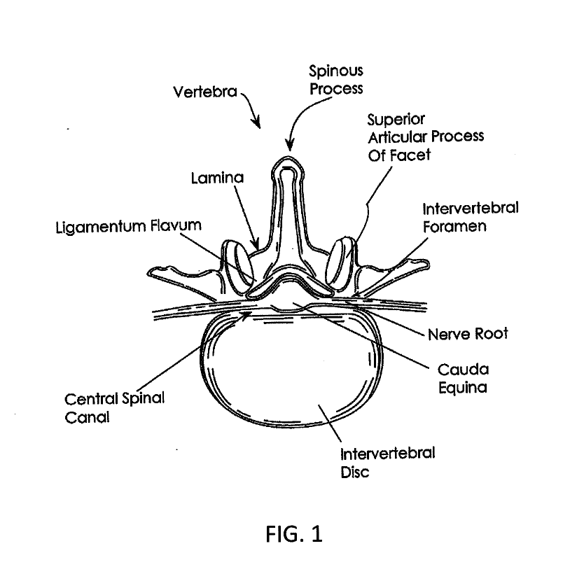

### DETAILED DESCRIPTION

Various embodiments of tissue modification devices and systems, as well as methods for making and using tissue modification devices and systems, are provided herein. In general, a curved tissue-modification device as described herein is configured to remove tissue from a patient. In particular, these tissue-modification devices may be configured to decompress spinal stenosis. These devices typically include a curved elongate body that extends proximally to distally (proximal/distal), and is configured to be inserted into a patient so that it extends around the target tissue, so that it can be pulled up against the target tissue. Thus, the device may be extended into, through, and/or around a spinal foramen. For example, in variations in which the device has an elongated, and in some embodiments, ribbon shape that is long and flat with a width greater than the thickness, the device includes a first major surface (e.g., a front) and a second major surface (a back), and has edges (minor surfaces) between the first and second major surfaces. The first major surface may be referred to as the anterior or front surface and the second major surface may be referred to as the posterior or back surface. The devices described herein may be flexible along the anterior and posterior surfaces, and the anterior or front surface may include one or more cutting edges configured to cut tissue as the anterior surface of the device is urged against a tissue. The posterior surface may be configured to shield or protect non-target tissue.

In general, these devices may be configured to be sufficiently stiff so that they may be pushed against the tissue to be cut (modified) and allow the device to grasp and modify the tissue. Some variations of these devices may be configured so that they may be made flexible for positioning in the tissue and later rigidified or stiffened so that they may be pushed/pulled against the tissue to be modified.

The devices described herein may include one or more tissue cutting members (e.g., a pair of biting/cutting members that may be used to cut the tissue. In some variation the biting/cutting members may be configured so that they are manually actuated to cut tissue. In some variations the biting/cutting members may be configured so that they are powered (e.g., mechanically, electrically, etc.) and may be driven against the tissue.

In general, the devices described herein may include a distal region that is relatively narrow or thin, allowing the device to be positioned even within the relatively tight or difficult to access regions such as the spine. For example, the devices described herein may be referred to as low-profile (e.g., less than 5 mm, less than 4 mm, less than 3 mm, less than 2 mm, etc.) or ultra low-profile (e.g., less than 3 mm, less than 2.5 mm, less than 2 mm, less than 1 mm, etc.). The “profile” typically refers to the height/thickness of the device. In contrast, the device may be relatively wider than they are high (e.g., 15 mm wide, 12 mm wide, 10 mm wide, 7 mm wide, 5 mm wide, etc.).

Although much of the following description and accompanying figures generally focuses on surgical procedures in spine, in alternative embodiments, devices, systems and methods of the present invention may be used in any of a number of other anatomical locations in a patient's body. For example, in some embodiments, the tissue modification devices of the present invention may be used in minimally invasive procedures in the shoulder, elbow, wrist, hand, hip, knee, foot, ankle, other joints, or other anatomical locations in the body. Similarly, although some embodiments may be used to remove or otherwise modify ligamentum flavum and/or bone in a spine to treat spinal stenosis, in alternative embodiments, other tissues may be modified to treat any of a number of other conditions. For example, in various embodiments, treated tissues may include but are not limited to ligament, tendon, bone, tumor, cyst, cartilage, scar, osteophyte, inflammatory tissue and the like. Non-target tissues may include neural tissue and/or neurovascular tissue in some embodiments or any of a number of other tissues and/or structures in other embodiments. In one alternative embodiment, for example, a flexible tissue modification device may be used to incise a transverse carpal ligament in a wrist while inhibiting damage to the median nerve, to perform a minimally invasive carpal tunnel release procedure. Thus, various embodiments described herein may be used to modify any of a number of different tissues, in any of a number of anatomical locations in the body, to treat any of a number of different conditions.

FIG. 2 is a top view of a vertebra with the cauda equina (the bundle of nerves that extends from the base of the spinal cord) shown in cross section and two nerve roots branching from the cauda equina to exit the central spinal canal and extend through intervertebral foramina on either side of the vertebra. Spinal stenosis can occur when the spinal cord, cauda equina and/or nerve root(s) are impinged by one or more tissues in the spine, such as buckled or thickened ligamentum flavum, hypertrophied facet joint, osteophytes (or “bone spurs”) on vertebrae, spondylolisthesis (sliding of one vertebra relative to an adjacent vertebra), facet joint synovial cysts, and/or collapse, bulging or herniation of an intervertebral disc. As shown in FIG. 2, the red tissue may be the impinging tissue. Impingement of neural and/or neurovascular tissue in the spine by one or more of these tissues may cause pain, numbness and/or loss of strength or mobility in one or both of a patient's lower limbs and/or of the patient's back.

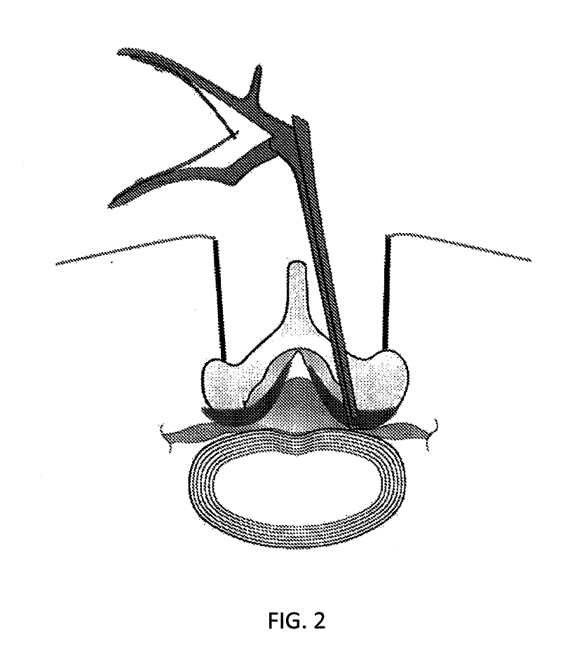

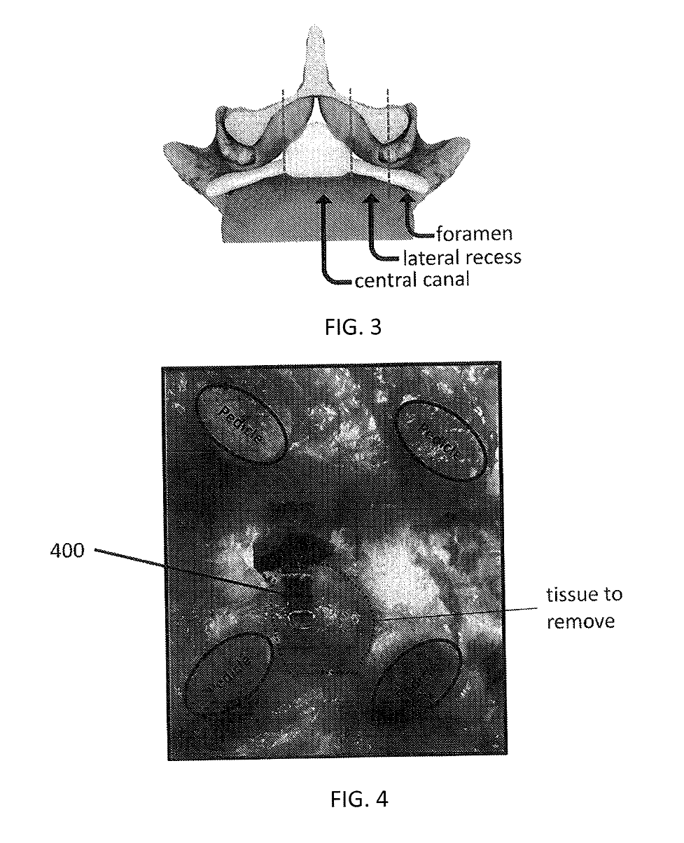

As described above, conventional lumbar spinal stenosis surgery involves first making an incision in the back and stripping muscles and supporting structures away from the spine to expose the posterior aspect of the vertebral column. Thickened ligamentum flavum is then exposed by complete or partial removal of the bony arch (lamina) covering the back of the spinal canal (laminectomy or laminotomy). As shown, conventional large, straight, rigid tools, such as rongeurs or bone punches are brought into the spine to attempt to remove the impinging tissue. As shown, due to their size and shape, conventionally tools are unable to access the impinging tissue in the lateral recess and foraminal regions (as shown in FIG. 3) and are therefore not able to perform a complete decompression. In an attempt to remove more of the impinging tissue, the surgery often includes partial or complete facetectomy (removal of all or part of one or more facet joints), to remove impinging ligamentum flavum or bone tissue.

FIG. 3 is a top view of a vertebra shown in cross section showing the central canal, lateral recess, and foraminal regions. Patients suffering from lumbar spinal stenosis may have impinging tissue in just the central canal, the lateral recess, or the foraminal region of one or more vertebra. Alternatively, a patient may have impinging tissue in a combination of regions, for example central with lateral recess stenosis, lateral recess with foraminal stenosis, all three regions or any other combination. As shown in FIG. 2, conventional tools are physically unable to remove tissue from the lateral recess and especially the foraminal region. Furthermore, if a patient has the majority of their stenosis in the lateral recess, it may be desirable to decompress only the lateral recess rather than decompressing the central canal and/or the foraminal region.

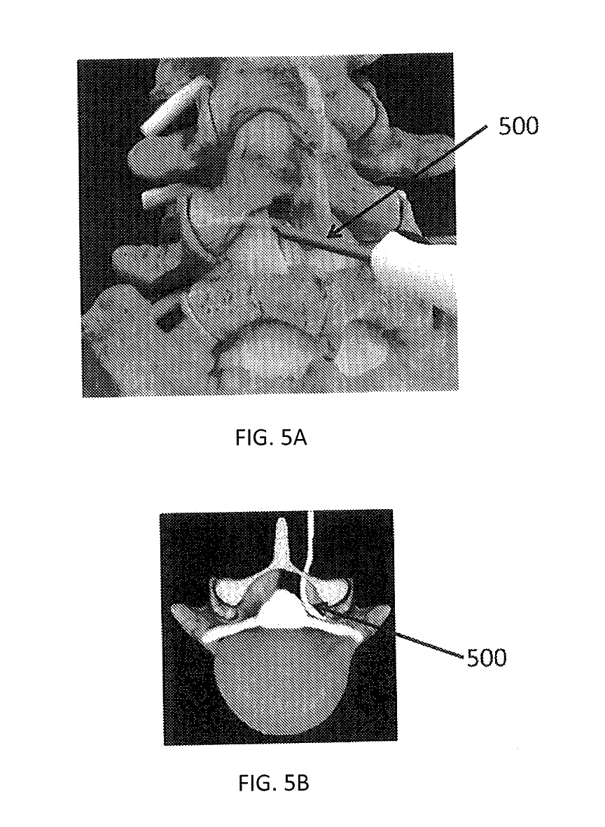

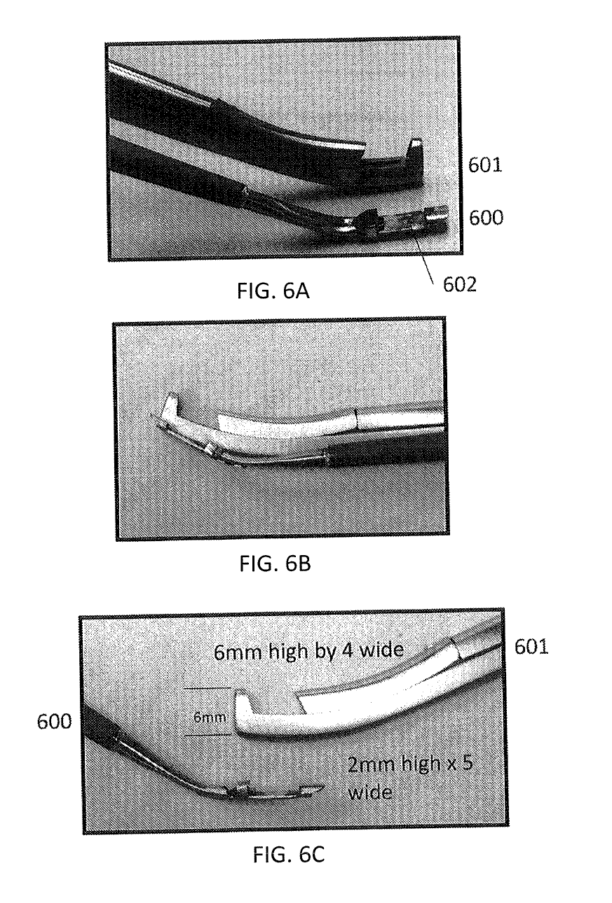

As shown in FIG. 4, the devices described herein may remove tissue from the lateral recess while not significantly removing tissue far out in the foraminal region. As shown in FIGS. 6 through 10B, the elongate body of the device may be sized and configured to not extend far out into the foramen if not desired. However, in some embodiments, the devices described herein may decompress foraminal stenosis as well. FIG. 4 is an anterior view of two vertebrae. The two pedicles of each vertebra are highlighted. As shown, device 400 has been placed through an interlaminar window, for example, and advanced from the central canal toward the foramen (between two pedicles), such that the lateral recess can be decompressed. As shown, ligamentus tissue and some bony tissue in the lateral recess may be removed with device 400. As shown in more detail in FIGS. 5A and 5B, device 500 may be advanced through an interlaminar window and around a facet joint. Once in position, the at least partially rigid device may be pulled up against the soft and bony tissue, within the lateral recess for example, to capture and remove tissue as described below.

Removal of vertebral bone, as occurs in laminectomy and facetectomy, often leaves the affected area of the spine very unstable, leading to a need for an additional highly invasive fusion procedure that puts extra demands on the patient's vertebrae and limits the patient's ability to move. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Thus, while laminectomy, facetectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients.

Described herein are tissue modification devices and methods for removing target impinging tissue while sparing healthy tissue. FIGS. 6A through 10 illustrate exemplary curved tissue modification devices for removing impinging tissue. As shown in FIG. 6A, a curved, thin profile tissue modification device 600 is compared to a conventional surgical rongeur 601. As shown, the device 600 includes an elongate body 602, having an axial length, a width and a thickness, wherein the axial length is greater than the width and the width is greater than the thickness. As shown, the thickness of the device 600 is substantially thinner than the conventional rongeur 601. As shown, the rongeur 601 may have a thickness or height between 4 and 10 mm. In one specific example, as shown in FIG. 6C, the rongeur may have a height of about 6 mm. As shown, the device 600 may have a height of only about 2 mm. In some embodiments the device thickness may be between 0 and 4 mm. In some embodiments, the width of the elongate body 602 of device 600 may be between 1 and 15 mm. In some embodiments, the width of the elongate body may be between 2 and 8 mm, while in some embodiments, the width may be between 3 and 5 mm. In one particular example, the width of the elongate body may be about 4 mm.

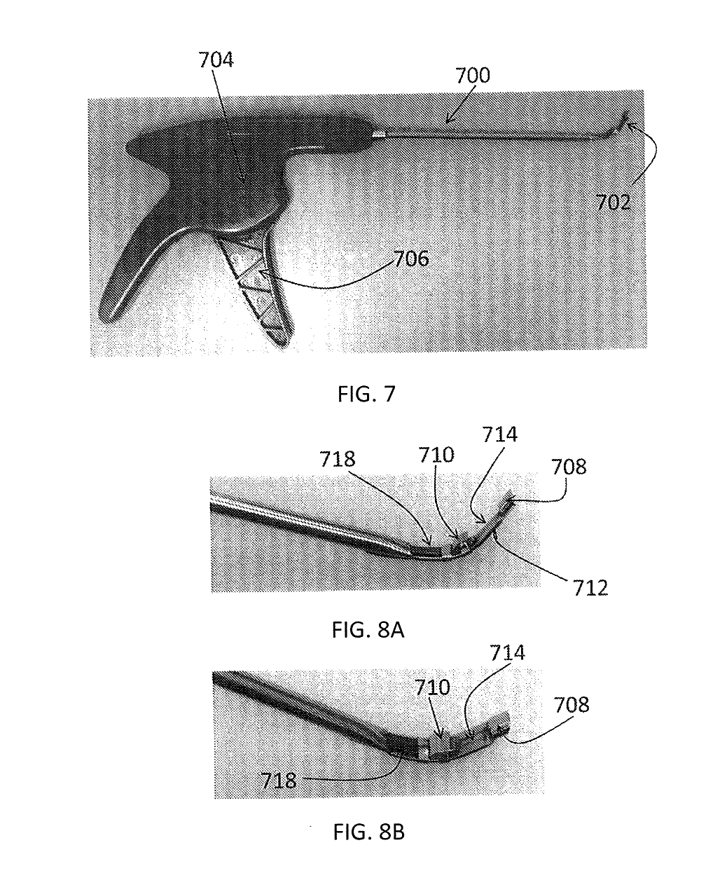

As shown in FIG. 7, the device 700 may include an elongate body 702, a handle 704 with an actuator 706, one or more tissue modifying members 708 and 710, and one or more protective surfaces 712. In some embodiments, the elongate body may further include a rigid shaft that couples the distal portion of the elongate body to the handle. In some embodiments, as shown in FIG. 7, a distal portion of the device is curved such that there is an angle between the rigid shaft and the distal portion of the elongate body. In some embodiments, the angle is between 180 degrees and 90 degrees, while in some embodiments, the angle is less than 90 degrees. In the embodiment shown, the tissue modifying members comprise blades, although in alternative embodiments other tissue modifying members may be added or substituted. Tissue modification via tissue modifying members may include cutting, ablating, dissecting, repairing, reducing blood flow in, shrinking, shaving, burring, biting, remodeling, biopsying, debriding, lysing, debulking, sanding, filing, planing, heating, cooling, vaporizing, delivering a drug to, and/or retracting the target tissue. In some embodiments (not shown), the device may further include a guidewire coupler at the distal end. The guidewire coupler may be configured to couple to a guidewire such that a guidewire may be removably coupled to the distal end of the device. The guidewire may be used to position and/or apply a distal tensioning force to the device to aid in tissue capture and removal.

In various embodiments, elongate body 702 may have any number of dimensions, shapes, profiles and amounts of flexibility or rigidity. In various embodiments, elongate body 108 may have one or more of a round, ovoid, ellipsoid, flat, cambered flat, rectangular, square, triangular, symmetric or asymmetric cross-sectional shape. As shown in FIGS. 8A and 8B, in the pictured embodiment, elongate body 702 may have a relatively flat configuration, which may facilitate placement of body 702 between target and non-target tissues. Distal portion of body 702 may be thin and/or tapered, to facilitate its passage into or through narrow spaces as well as through small incisions on a patient's skin. Body 702 may also include a slightly widened portion around the area of window 714 and blades. In some embodiments, the window or portion of device between the blades may be curved. Alternatively, as shown in FIGS. 8A and 8B, the window or portion of device between the blades may be straight. In one embodiment, such as an embodiment used for modifying tissue in a spine, body 702 may have a small profile, such as having a height of not more than 10 mm at any point along its length and a width of not more than 20 mm at any point along its length, or more preferably a height not more than 5 mm at any point along its length and a width of not more than 10 mm at any point along its length, or even more preferably a height not more than 2 mm at any point along its length and a width of not more than 4 mm at any point along its length. Body 702 may be long enough to extend through a first incision on a patient, between target and non-target tissue, and out a second incision on a patient. Alternatively, body 702 may be long enough to extend through a first incision, between the target and non-target tissue, and to an anchoring location within the patient. In another alternative embodiment, body 702 may be long enough to extend through a first incision, between the target and non-target tissue, to a location nearby but distal to the target tissue within the patient, with some portion of tissue modification device 700 anchored to a guidewire (as described above, not shown). In some embodiments, elongate body 702 includes at least one feature for allowing passage of the body over a guidewire or other guide member or to allow passage of one or more guide members over or through body 702. For example, in various embodiments, body 702 may include one or more guidewire lumens, rails, tracks, lengthwise impressions or some combination thereof.

In some embodiments, it may be advantageous to include one or more rigid sections in elongate body 702, such as to impart pushability to a portion of body 702 or to facilitate application of force to tissue modification members 708 and 710 without causing unwanted bending or kinking of elongate body 702. In such embodiments, rigidity may be conferred by using additional materials in body 702 or by making the rigid portions thicker or wider or of a different shape.

Handle 704 may have any suitable configuration according to various embodiments. Similarly, actuator 706 may include any of a number of actuation devices in various embodiments. In the embodiment shown in FIG. 7, actuator 706 comprises a trigger or moving handle portion, which is grasped by a user and pulled or squeezed toward handle 704 to bring blades 708 and 710 together to cut tissue. In an alternative embodiment, actuator 706 instead may include a switch or button for activating a radiofrequency surgical ablation tissue modifying member. In yet another embodiment, actuator 106 may include a combination trigger and switch, one or more pull wires, any suitable form of lever and/or some combination thereof.

FIGS. 8A and 8B show in greater detail a portion of tissue modification device 700. In these figures, window 714 and blades 708 and 710 are more clearly seen. In one embodiment, as shown, at least a portion of elongate body and blades may have a slightly curved configuration. In alternative embodiments, at least a portion of elongate body and blades may be flat. In other alternative embodiments, tissue modification members such as blades may be proud to the elongate body.

Blades 708 and 710 include a distal 708 and a proximal blade 710 that reside at the distal and proximal edges, respectively, of window 714 of elongate body 702. The window may accommodate both soft and hard tissue when the device is forcibly applied to the surface of a target tissue site. In some embodiments, the blades may include the angled edges, which facilitate shearing of target tissue. In alternative embodiments, the blades may have any of a number of alternative shapes and configurations. In some embodiments, the distal portion of body 702 may have a very low profile (height compared to width), as shown in side view FIGS. 6A through 8B, where only the blades protrude from the top surface of the elongate body. In some embodiments, the lower surface of elongate body is an example of a protective or non-tissue-modifying surface 712.

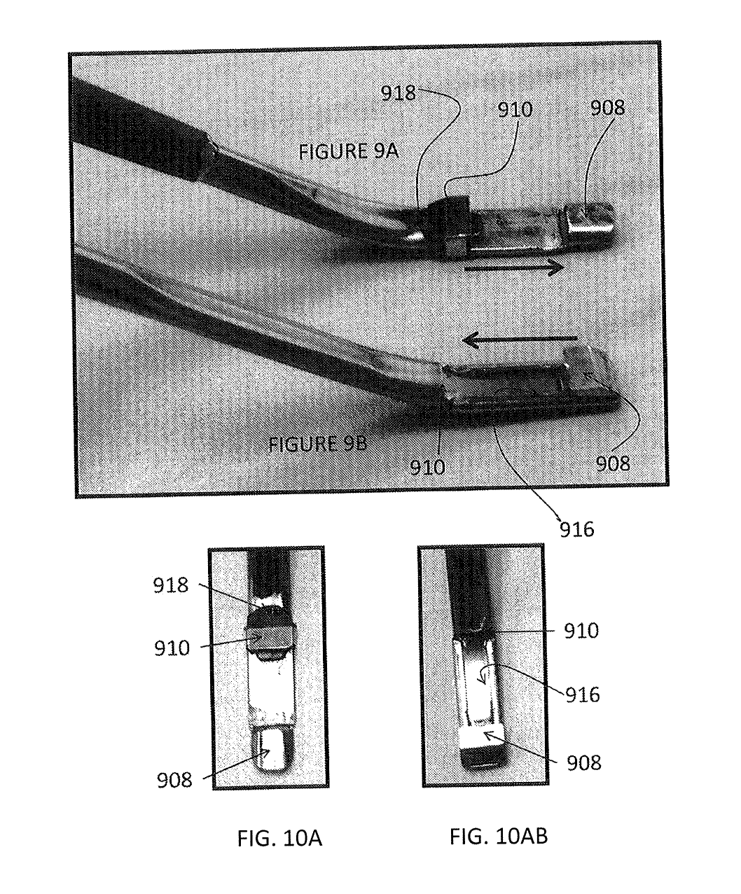

In one embodiment, as shown in FIGS. 9A and 10A, proximal blade 910 may be coupled with a push mechanism, such as the multi-wire drive mechanism 918 (see also reference 718 in FIGS. 8A and 8B). Drive mechanism 918 may be coupled to and translated by actuator 706 on handle 704 and may be used to drive or push proximal blade 710 distally to contact the cutting edge of distal blade 708, thus cutting tissue. In one embodiment, as shown in FIGS. 9B and 8B, distal blade 908 may be coupled with a pull mechanism, such as two pull-wires 916 (shown within a channel or guide). Pull-wires may be coupled to and translated by actuator 706 on handle 704 and may be used to drive or pull distal blade 708 proximally to contact the cutting edge of proximal blade 710, thus cutting tissue. In some alternative embodiments, the distal blade 708 may be pulled and the proximal blade 710 may be pushed such that each blade moves toward the opposite blade to cut.

Other alternative mechanisms for driving blades, such as gears, ribbons or belts, magnets, electrically powered, shape memory alloy, electromagnetic solenoids and/or the like, coupled to suitable actuators, may be used in alternative embodiments. As mentioned, in one embodiment distal blade and/or proximal blade may have an outwardly curvilinear shape along its cutting edge. Alternatively, distal blade may have a different blade shape, including flat, rectilinear, v-shaped, and inwardly curvilinear (concave vs. convex). The cutting edge of either blade 110 may have a sharp edge formed by a simple bevel or chamfer. Alternatively or in addition, a cutting edge may have tooth-like elements that interlock with a cutting edge of an opposing blade, or may have corrugated ridges, serrations, rasp-like features, or the like. In various embodiments, both blades 110 may be of equal sharpness, or alternatively one blade 110 may be sharp and the other substantially flat to provide a surface against which the sharp blade 110 may cut. Alternately or in addition, both cutting edges may be equally hard, or a first cutting edge may be harder than a second, the latter of which deflects under force from the first harder edge to facilitate shearing of the target tissue.

In some embodiments, all or a portion of elongate body, such as the lower surface 712, may include a lubricious surface for facilitating manipulation of the tool in the surgical space and at the anatomical site. The lubricious lower surface also provides a barrier between blades and non-target tissue in the surgical space

In some embodiments, when at least one of the blades is moved to cut tissue, at least some of the cut tissue may be captured in a hollow interior portion of elongate body. Various embodiments may further include a cover, a cut tissue housing portion and/or the like for collecting cut tissue and/or other tissue debris. Such collected tissue and debris may then be removed from the patient during or after a tissue modification procedure. During a given tissue modification procedure, distal blade, for example, may be drawn proximally to cut tissue, allowed to retract distally, and drawn proximally again to further cut tissue as many times as desired to achieve a desired amount of tissue cutting.

The blades may be made from any suitable metal, polymer, ceramic, or combination thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). In some embodiments, materials for the blades or for portions or coatings of the blades may be chosen for their electrically conductive or thermally resistive properties. Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. In various embodiments, blades may be manufactured using metal injection molding (MIM), CNC machining, injection molding, grinding and/or the like. Pull wires or drive mechanisms may be made from metal or polymer and may have circular, oval, rectangular, square or braided cross-sections.

Depending on the tissue to be treated or modified, activating blades (or other tissue modifying members in alternative embodiments) may cause them to modify target tissue along an area having any of a number of suitable lengths. In use, it may also be advantageous to limit the extent of action of blades or other tissue modifying members to a desired length of tissue, thus not allowing blades to affect tissue beyond that length. In so limiting the effect of blades, unwanted modification of, or damage to, surrounding tissues and structures may be limited or even eliminated. In one embodiment, for example, where the tissue modification device is used to modify tissue in a spine, blades may operate along a length of target tissue of no more than 10 cm, and preferably no more than 6 cm, and even more preferably no more than 3 cm. Of course, in other parts of the body and to address other tissues, different tissue modification devices may be used and tissue modifying members may have many different lengths of activity. In one embodiment, to facilitate proper location of tissue modifying members, such as blades, relative to target tissue, the tissue modifying members and/or the elongate body and/or one or more additional features intended for just such a purpose may be composed of a material readily identifiable via x-ray, fluoroscopic, magnetic resonance or ultrasound imaging techniques.

In various embodiments, a number of different techniques may be used to prevent blades 110 (or other tissue modifying members) from extending significantly beyond the target tissue. In one embodiment, for example, preventing blades 110 from extending significantly beyond the target tissue involves holding tissue modification device 102 as a whole predominantly stable to prevent device 102 from translating in a direction toward its proximal portion or toward its distal portion while activating blades 110. Holding device 102 stable is achieved by anchoring one end of the device and applying tensioning force at or near the other end, as described further below.

In some embodiments, pull wires may be retracted proximally by squeezing the actuator proximally. In an alternative embodiment, squeezing the actuator may cause both of the blades to translate inward so that they meet approximately in the middle of the window. In a further embodiment, the distal blade may be returned to its starting position by a pulling force generated from the distal end of the device, for example by using a distal actuator that is attached to distal wires, or by pulling on the distal guide member which is attached to the distal blade. In yet another alternative embodiment, the proximal blade may be moved to cut by a pulling force generated from the distal end of device, for example by using a distal actuator that is attached to distal wires, or by pulling on the distal guide member which is attached to proximal blade. In yet another embodiment, squeezing actuator may cause proximal blade to move distally while the distal blade stays fixed. In other alternative embodiments, one or more blades may move side-to-side, one or more blades may pop, slide or bow up out of the window when activated, or one or more blades may expand through window. In another embodiment, one or more blades and/or other tissue modifying members of device may be powered devices configured to cut, shave, grind, abrade and/or resect target tissue. In other embodiments, one or more blades may be coupled with an energy transmission device, such as a radiofrequency (RF) or thermal resistive device, to provide energy to blade(s) for cutting, ablating, shrinking, dissecting, coagulating or heating and thus enhancing tissue modification. In another embodiment, a rasp or file may be used in conjunction with or coupled with one or more blades. In any of these embodiments, use of actuator and one or more moving blades provides for tissue modification with relatively little overall translation or other movement of tissue modification device. Thus, target tissue may be modified without extending blades or other tissue modification members significantly beyond an area of target tissue to be treated.

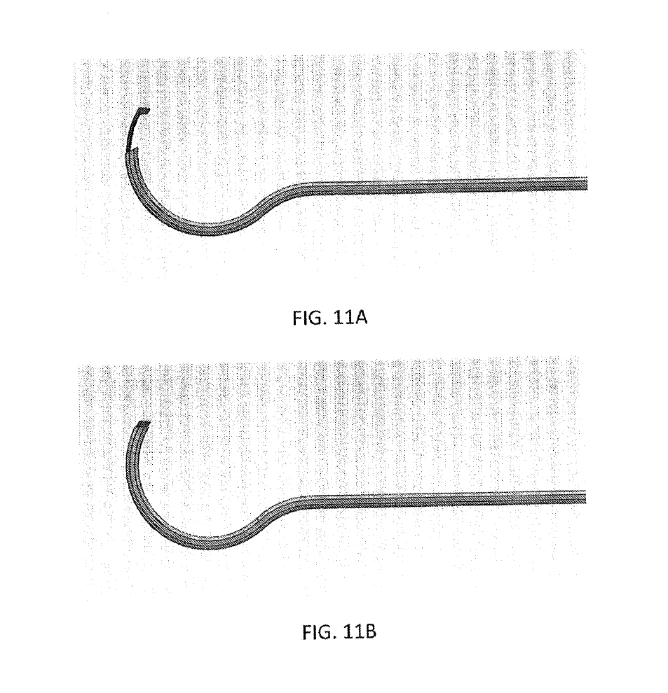

Described herein are tissue modification devices and methods for removing target impinging tissue while sparing healthy tissue. FIGS. 11A through 17B illustrate exemplary curved tissue modification devices for removing impinging tissue. As shown, in some embodiments, the tissue modification device may include a curved tube and a curved cutting member disposed within the curved tube. As shown, the distal end of the curved tube and cutting member may be configured to curve at least partially around target tissue such as a facet joint and/or ligamentum flavum. The curved distal end of the device may be advanced into the patient in a medial to lateral direction through an interlaminar window for example. The device may be passed through the interlaminar window and toward the lateral recess. In some embodiments, the device may be passed through the lateral recess toward a neural foramen. In some embodiments, the device may be passed through the neural foramen. In some embodiments, a laminotomy may be performed, and the device may be passed into the spine of a patient through the window created by the laminotomy. In some alternative methods, the device may be passed in a lateral to medial direction through a neural foramen. Once the device is in place, the device may be moved against the target tissue. The target tissue may include ligamentum flavum and/or the anterior aspect of the facet joint. In some alternative embodiments, the device may be used to remove disc material, bone spurs, etc. As shown in FIG. 11A for example, the device includes a window or bit opening between the distal cutting surface of the curved cutting member and the distal cutting surface of the curved tube. This opening may have any suitable dimensions. For example, this opening may be between 1 mm and 15 mm. In some embodiments, the bit opening may be between 3 mm and 10 mm. In one specific embodiment, the bit opening may be 7 mm. The device may be moved such that the bit opening is advanced over the target tissue to be removed. As shown in FIG. 11B, the curved tube may be advanced distally, such that it advances over the curved cutting member, and the distal cutting surface of the curved tube comes in contact with the distal cutting surface of the curved cutting member. As the tube is advanced and the blades or cutting surfaces come in contact with one another the target tissue within the bit opening will be cut and thereby removed. In some alternative embodiments, the cutting member could be pulled back proximally within the cutting tube. The cutting tube and/or cutting member may be made from a flexible and/or shape memory material such as Nitinol. In some embodiments, at least one of the cutting tube and cutting member may be rigid, such that the device can be pulled or pushed against the target tissue.

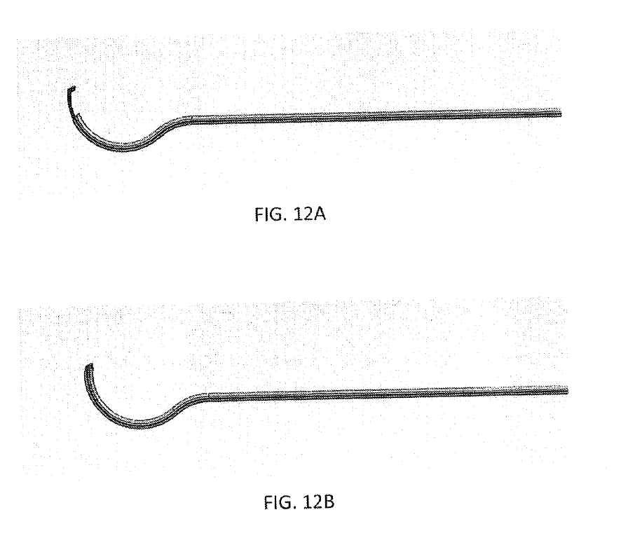

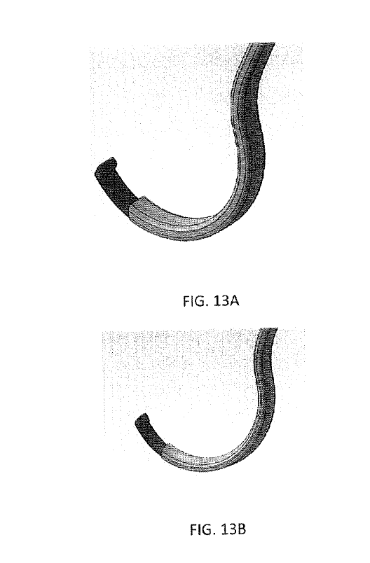

The curved tube and cutting member may be configured in any suitable curved shape. For example, the curve of the device as shown in FIGS. 12A and 12B, may have a reduced arc length as compared to the curve shape of the device embodiment as shown in FIGS. 11A and 11B. FIGS. 13 A and 13B illustrate in more detail the distal cutting surface of the cutting member, and the distal cutting surface of the curved tube. As shown, the distal cutting surface of the cutting member may function as the distal blade, and the distal cutting surface of the curved tube may function as the proximal blade. As discussed above, the distal blade may be pulled back against the proximal blade, the proximal blade may be pushed against the distal blade, or the two blades may both be moved toward one another. As shown in FIG. 13A, the cutting surfaces may have a rectangular shape. The rectangular shape of the cutting surfaces may be of any suitable size to adequately cut the target tissue. For example, the cutting surfaces may have a height between 0 mm and 10 mm. In one specific embodiment, the height may be 2.5 mm. The cutting surface may have a width between 1 mm and 10 mm. In one specific embodiment, the width may be 5 mm. As shown in FIG. 13B, the cutting surfaces may have a tombstone or curved shape. The tombstone shape of the cutting surfaces may be of any suitable size to adequately cut the target tissue. For example, the cutting surface may have a height between 0 mm and 10 mm. In one specific embodiment, the height may be 2.5 mm. The cutting surface may have a width between 1 mm and 10 mm. In one specific embodiment, the width may be 5 mm.

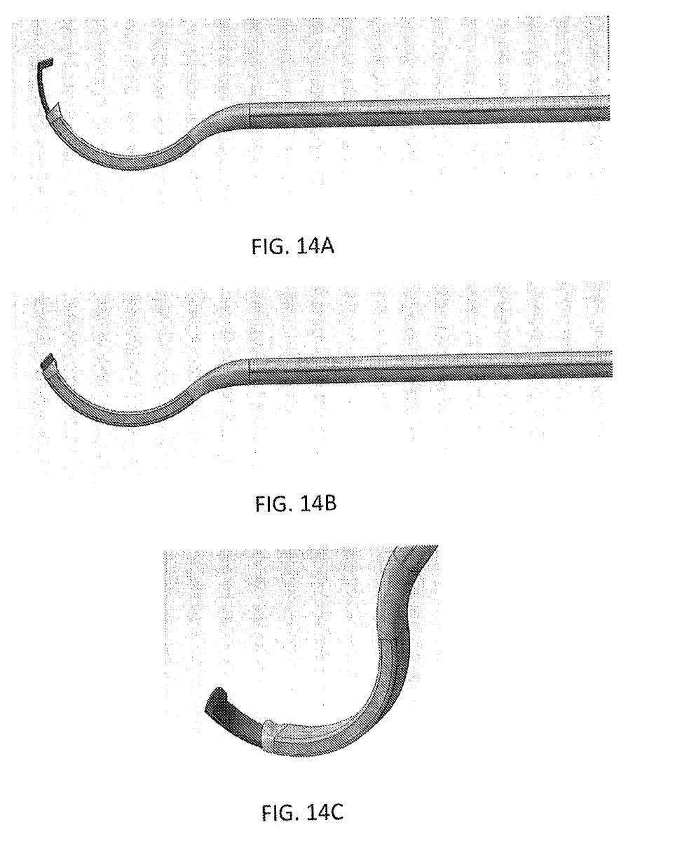

FIGS. 14A to 14C illustrate an alternative embodiment of the tissue modification device described herein. As shown, the proximal shaft of the tube and the transition portion of the tube between the shaft and the curved distal end have a larger diameter as compared to the embodiments as shown in FIGS. 11 and 12. The larger diameter may increase the stiffness of the device, and in some embodiments may better provide for holding the device against the target tissue to be modified. Furthermore, as shown in detail in FIG. 14C, the distal end of the cutting tube may include a cutting hood or otherwise enlarged cutting surface for increased tissue removal.

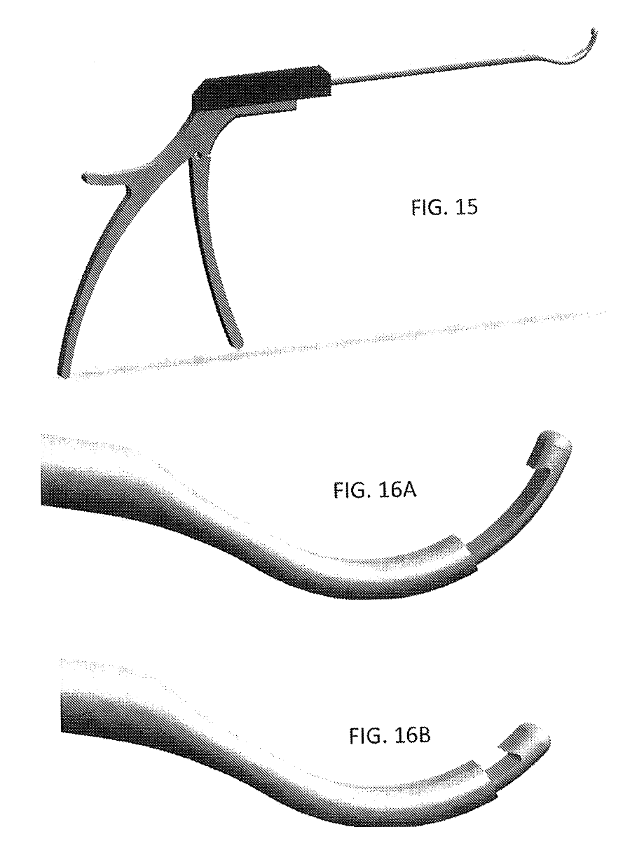

FIGS. 15 to 17B illustrate an alternative embodiment of the tissue modification device described herein. As shown in FIG. 15, the tissue modification device includes an actuation mechanism, such as the handle as shown. In some embodiments, when the lever of the handle is pulled back, the top slide portion will be moved forward, pushing the curved tube forward in the distal direction toward the distal blade of the cutting member. The inner cutting member may be fixed to the back handle portion. In some embodiments, the inner cutting member may be coupled directly, or may be coupled via wires or any other suitable coupling member.

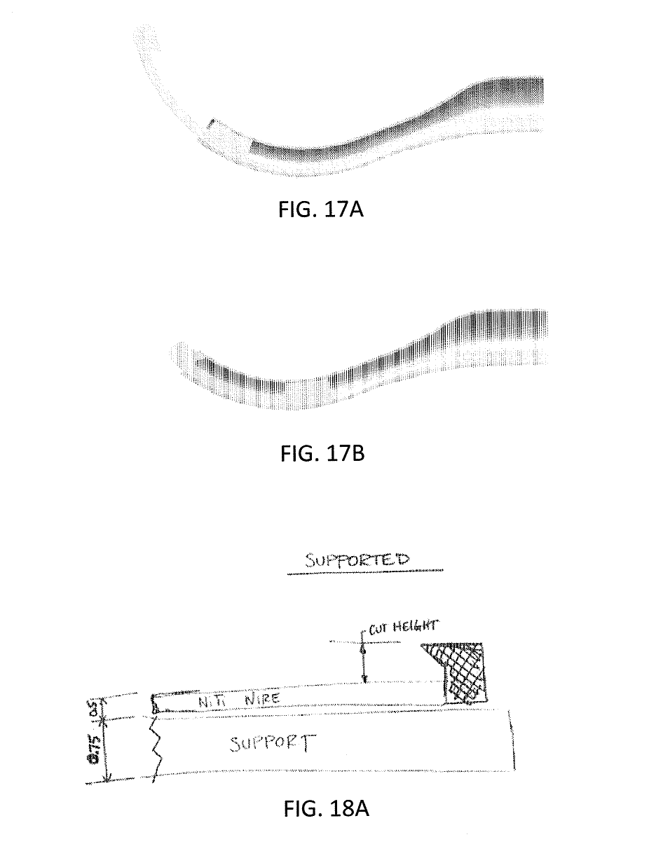

FIGS. 16A and 16B illustrate in more detail the distal end of the tissue modification device. As described above the device includes a tube member and a cutting member disposed within the tube member. The distal end of the tube member may function as the proximal blade, and the cutting surface of the cutting member may function as the distal blade. FIGS. 17A and 17B illustrate the distal end of the tissue modification device in cross section. The cutting member may be made of stainless steel or any other suitable material for cutting tissue. The tube member may be made of a flexible and/or shape memory material such that it may change shape as it is advanced over the cutting member. Alternatively, the curved tube may have a fixed shape and the cutting member may flex or shape change as the device is actuated. For example, as shown in FIGS. 17A and 17B, the inner cutting member may be coupled to flexible or shape changing wires (not shown) that may flex and/or shape change as the tube is advanced over the wires. As shown in FIG. 17B as the tube is advanced over the cutting member (or as the cutting member is pulled into the tube), the cutting member fits within the tube and/or conforms within the tube. The wires (not shown) may flex and bend to accommodate the curved tube while the cutting member may remain rigid.

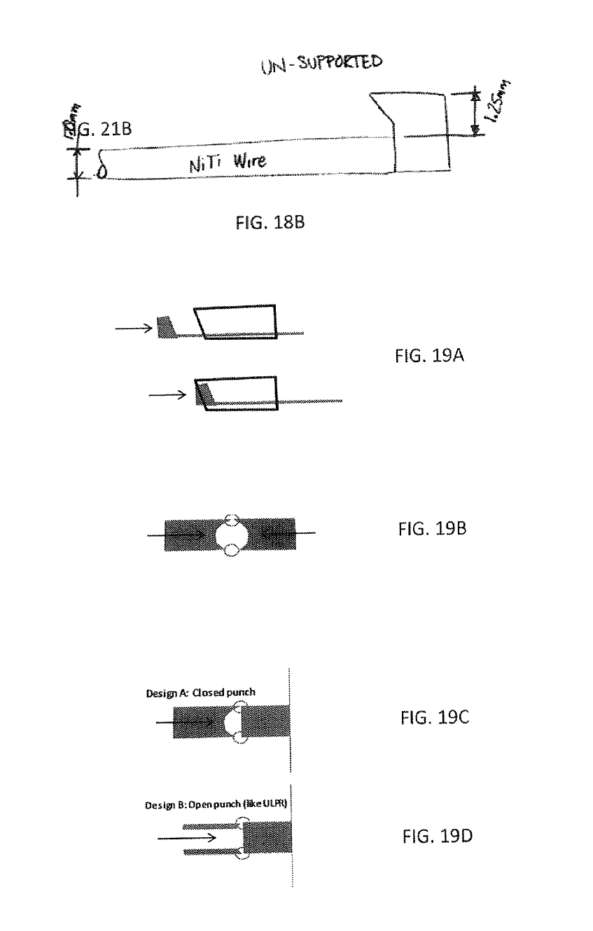

FIGS. 18A and 18B, illustrate a supported embodiment and a non-supported embodiment, respectively. The embodiments of FIGS. 18A and 18B are both “pull” embodiments, wherein the distal blade is pulled back against a proximal blade (not shown). In some embodiments, as shown in FIG. 18B, the material and shape combinations may allow the blade and Nitinol wire to function appropriately without a support member. Alternatively, as shown in FIG. 18A the device may not include a support member.

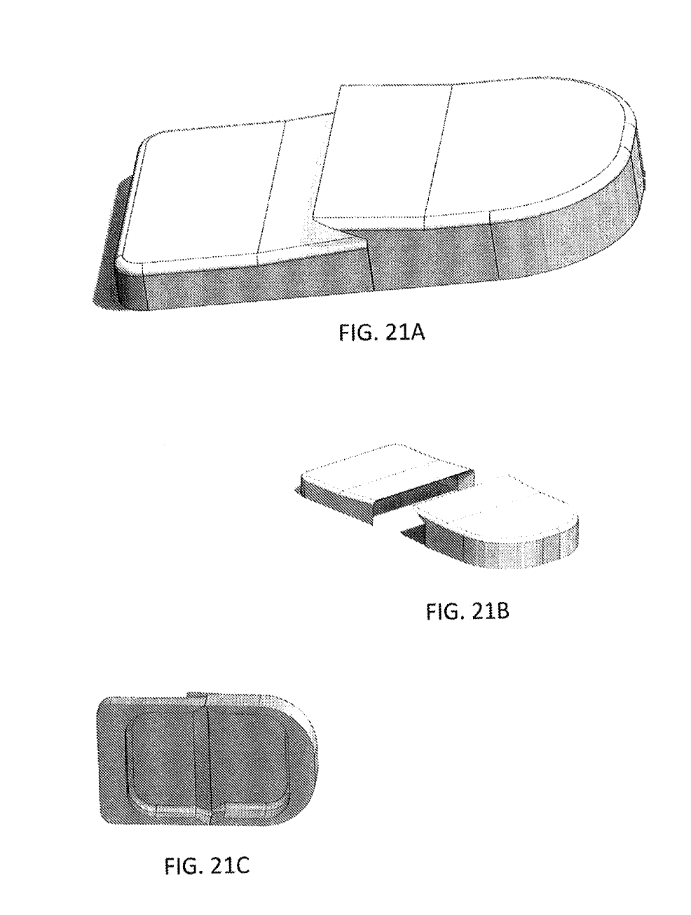

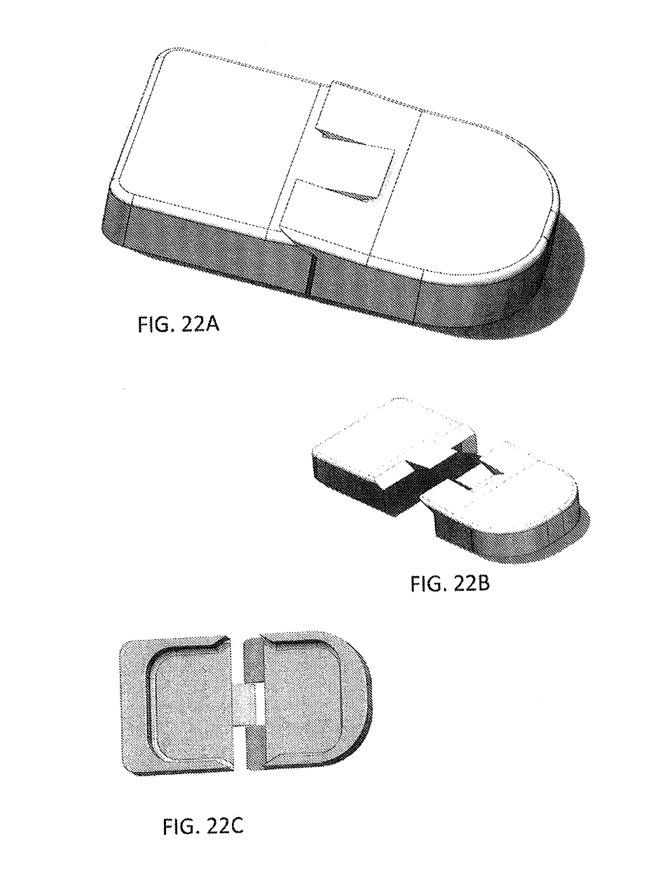

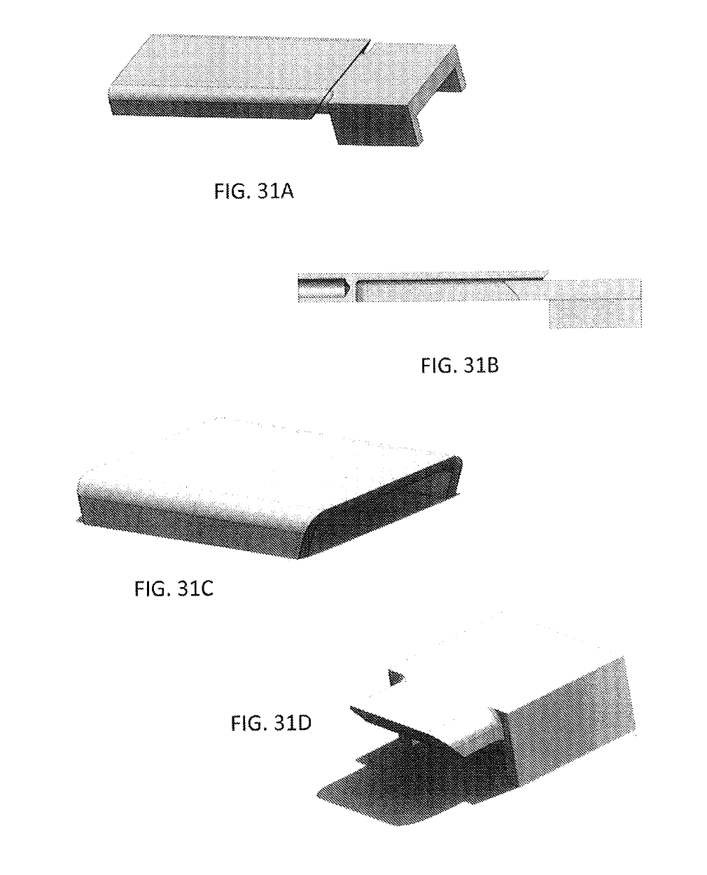

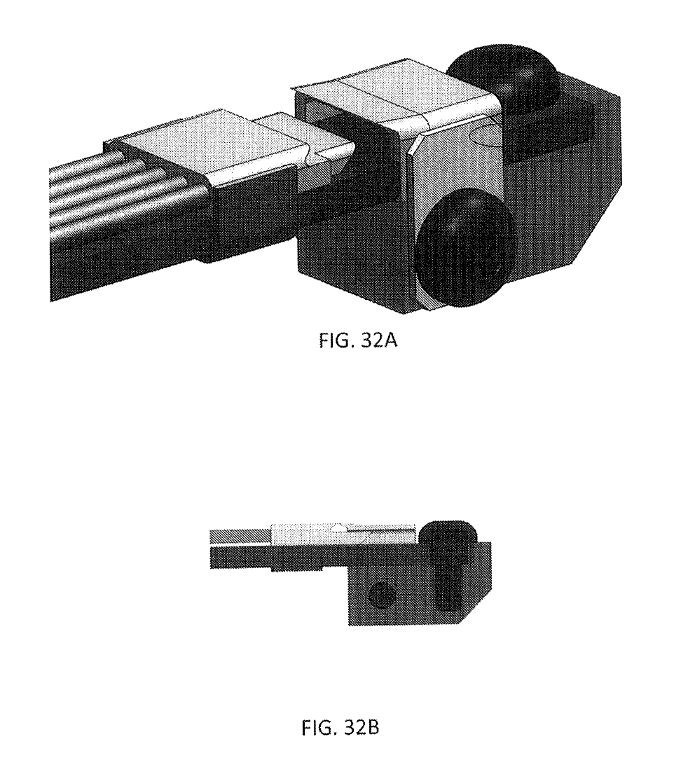

FIGS. 19A to 32B illustrate various blade embodiments and blade combinations. As shown in FIGS. 19A through 19D, the blades of the tissue modification device may cut in one of several different mechanisms. For example, as shown in FIG. 19A, the blades may cut with a shear cutting mechanism. Conventional scissors typically utilize a shear cutting mechanism. As shown in FIG. 19B, the cutting mechanism may be a pinch cutting mechanism. Alternatively, as shown in FIGS. 19C and 19D, the cutting mechanism may be a punch mechanism. In some embodiments, as shown in FIG. 19C, the punch may be open, while in other embodiments, the punch may be a closed punch, as shown in FIG. 19D. Any of these cutting mechanisms may be used in a pull configuration, a push configuration, or a push/pull configuration as described above.

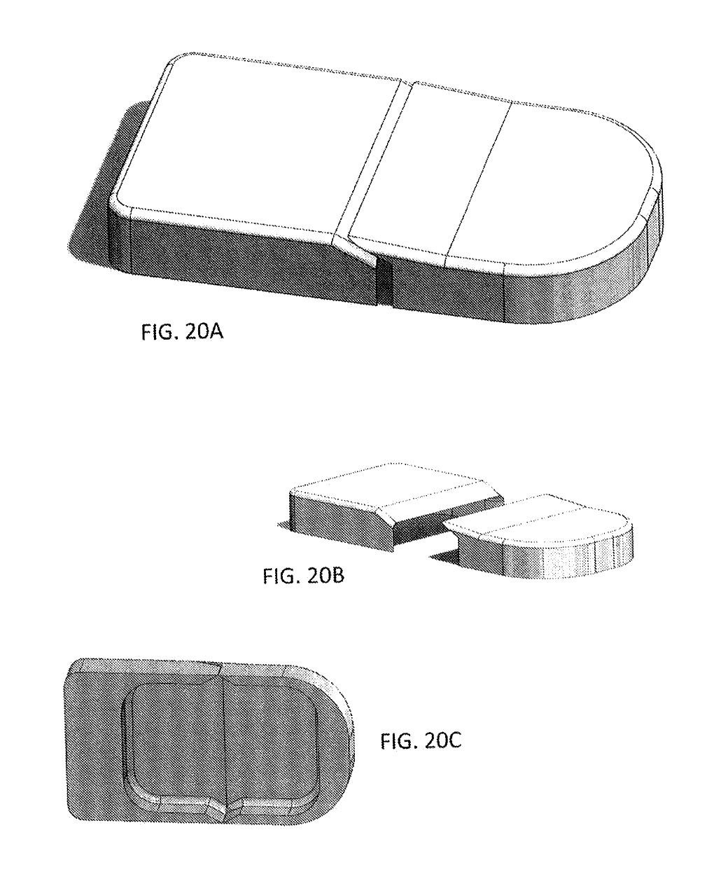

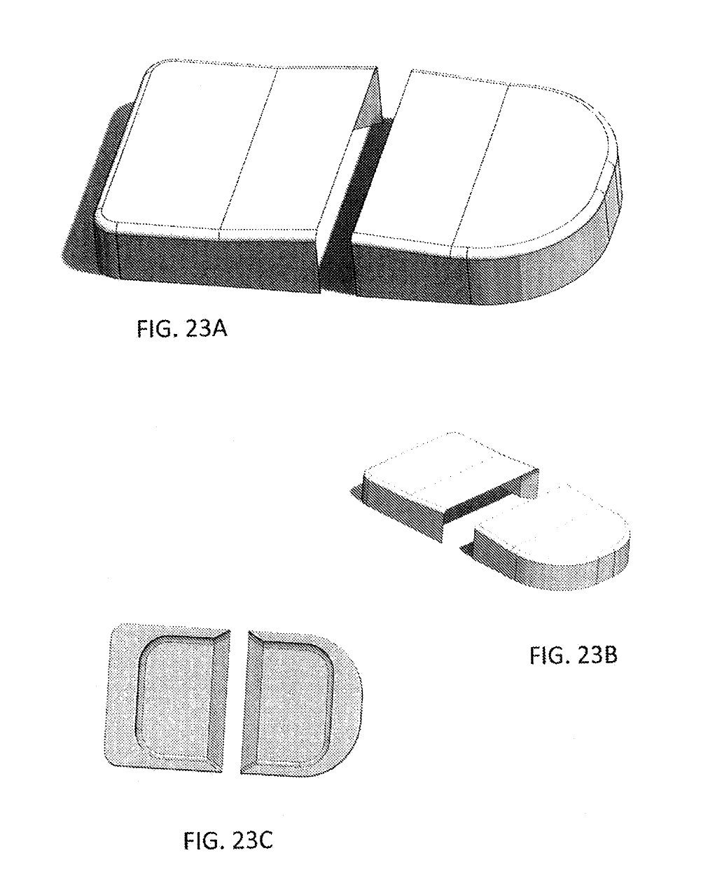

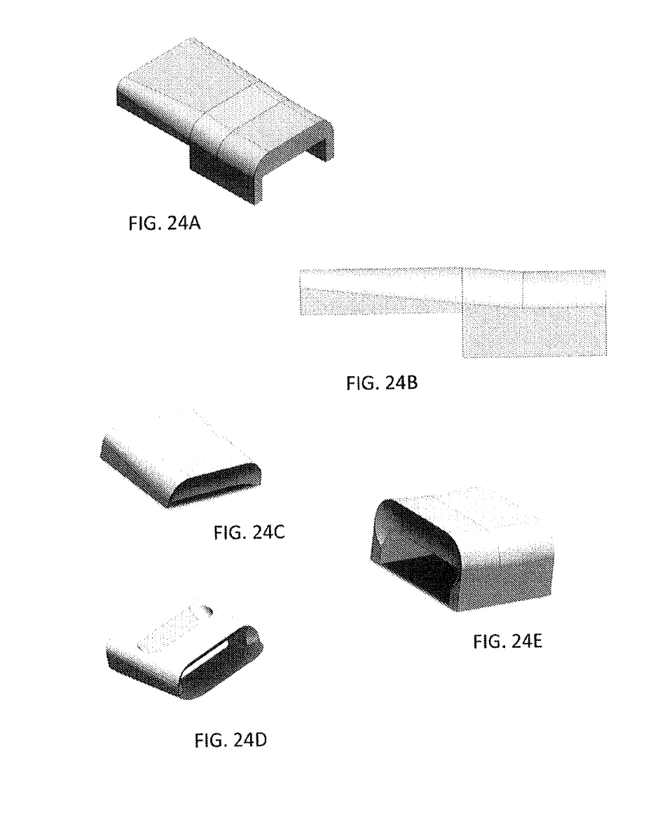

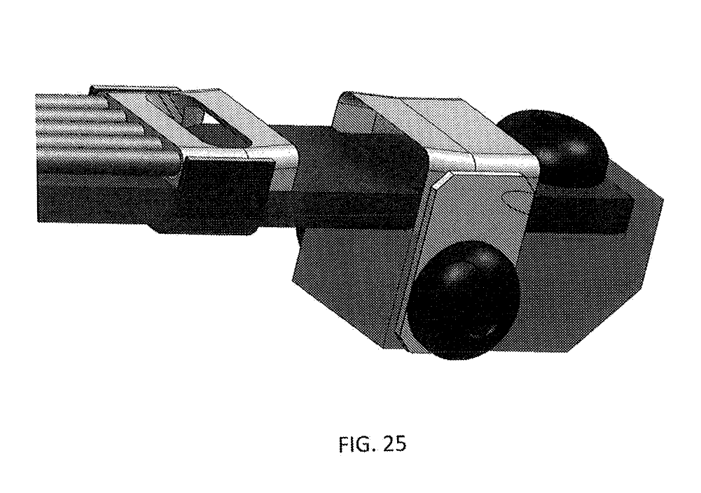

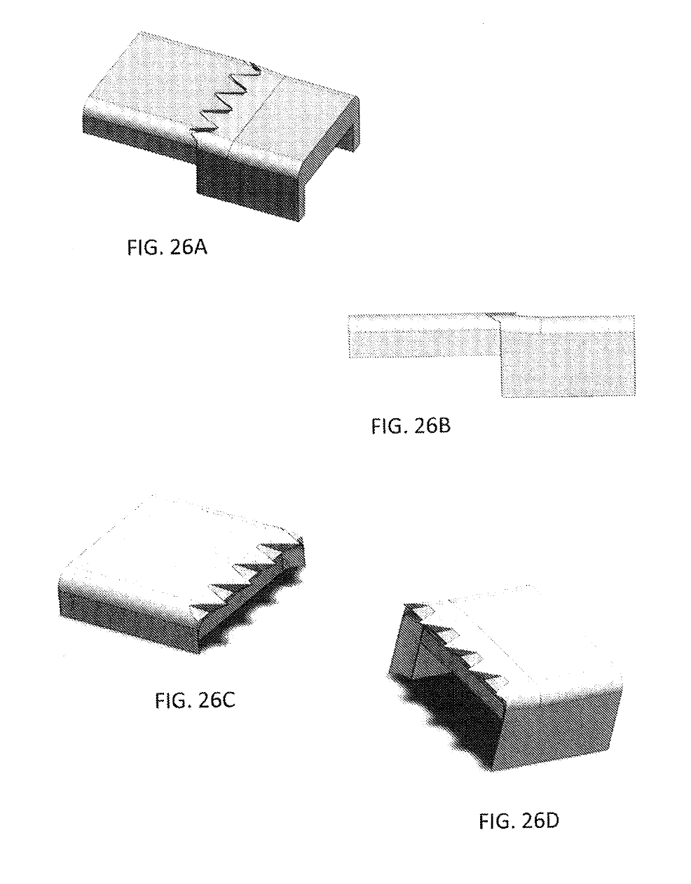

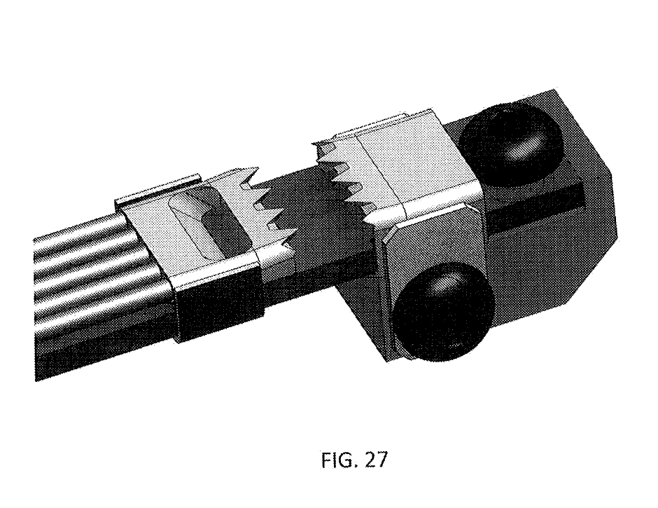

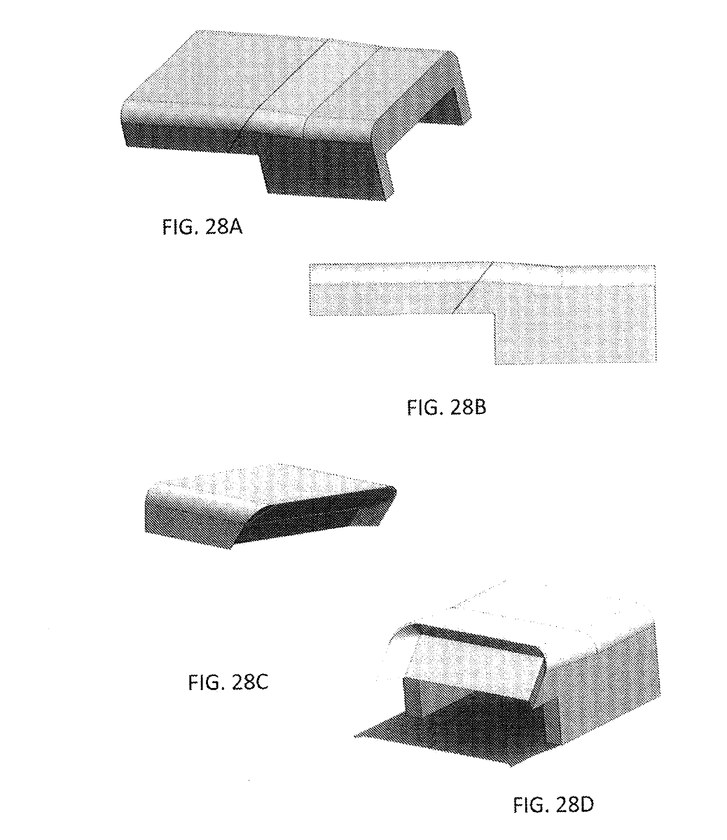

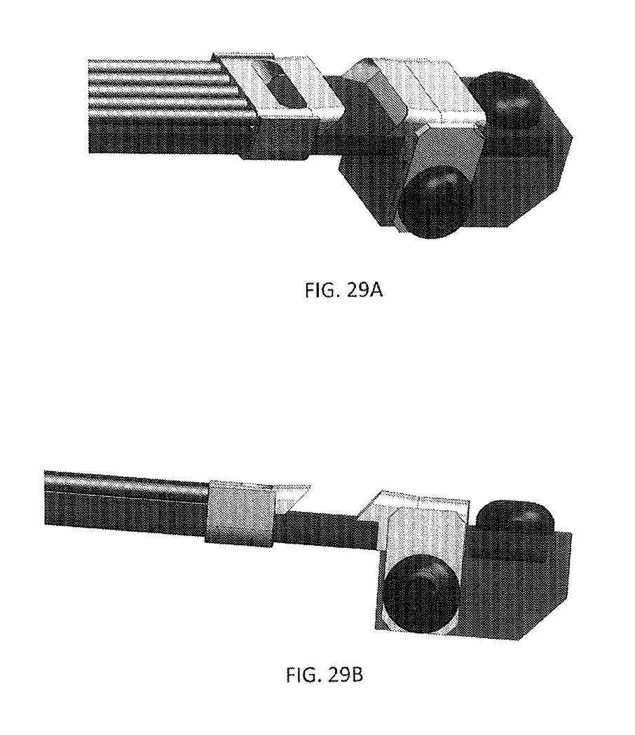

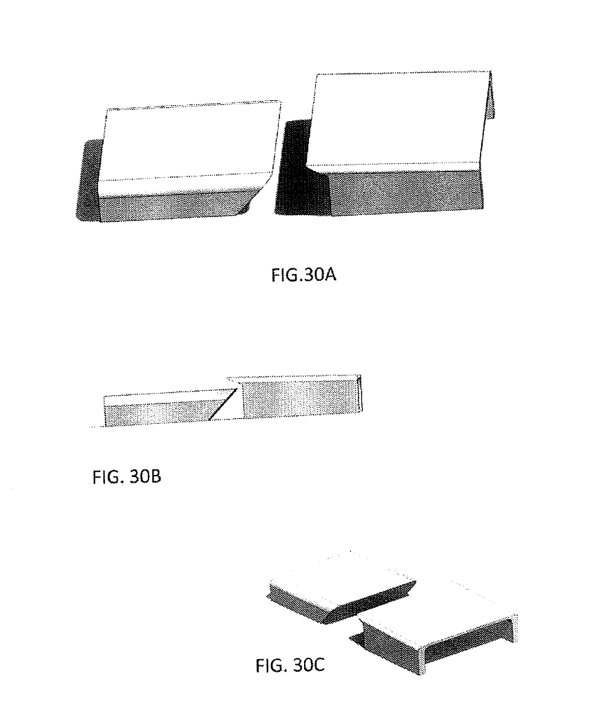

FIGS. 20A through 29B illustrate examples of blades utilizing a pinching mechanism. FIGS. 30A through 32B illustrate examples of blades utilizing a shear cutting mechanism. In one specific example, as shown in FIGS. 23A to 25, the blades may be a 90 degree pinch blade set. FIG. 24D illustrates that the proximal blade may include an opening through which cut tissue may pass. Tissue management is discussed in more detail below. FIG. 25 illustrates the mounting of the blades to a tissue modification device. In some embodiments, at least one wire may be coupled to the proximal blade. The wire may be flexible and/or shape changing such that it can flex or bend to accommodate the curve of the outer tube or support member as described about. In some embodiments, the proximal blade may be pushed distally toward the distal blade. Alternatively, the distal blade may be pulled proximally toward the proximal blade. In another specific example, as shown in FIGS. 26A to 27, the blades may be a 90 degree pinch blade set having cutting teeth disposed along the cutting surfaces of the blades. FIG. 27 illustrates the mounting of the blades to a tissue modification device. In some embodiments, at least one wire may be coupled to the proximal blade. The wire may be flexible and/or shape changing such that it can flex or bend to accommodate the curve of the outer tube or support member as described about. In some embodiments, the proximal blade may be pushed distally toward the distal blade. Alternatively, the distal blade may be pulled proximally toward the proximal blade. In another specific example, as shown in FIGS. 28A to 29B, the blades may be a 40 degree angled pinch blade set. FIGS. 29A and 29B illustrate that the proximal blade may include an opening through which cut tissue may pass. Tissue management is discussed in more detail below. FIGS. 29A and 29B illustrate the mounting of the blades to a tissue modification device. In some embodiments, at least one wire may be coupled to the proximal blade. The wire may be flexible and/or shape changing such that it can flex or bend to accommodate the curve of the outer tube or support member as described about. In some embodiments, the proximal blade may be pushed distally toward the distal blade. Alternatively, the distal blade may be pulled proximally toward the proximal blade.

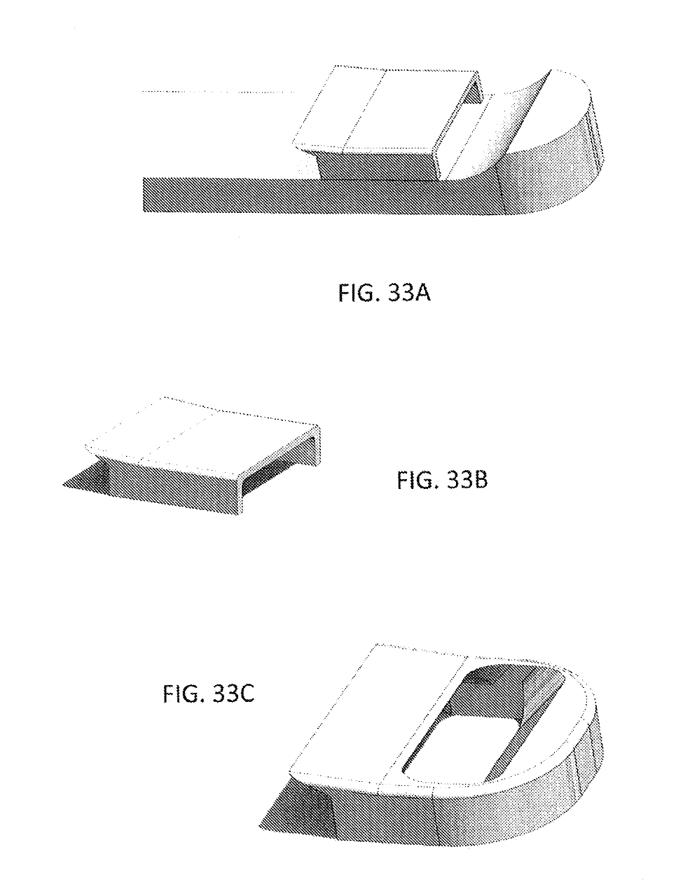

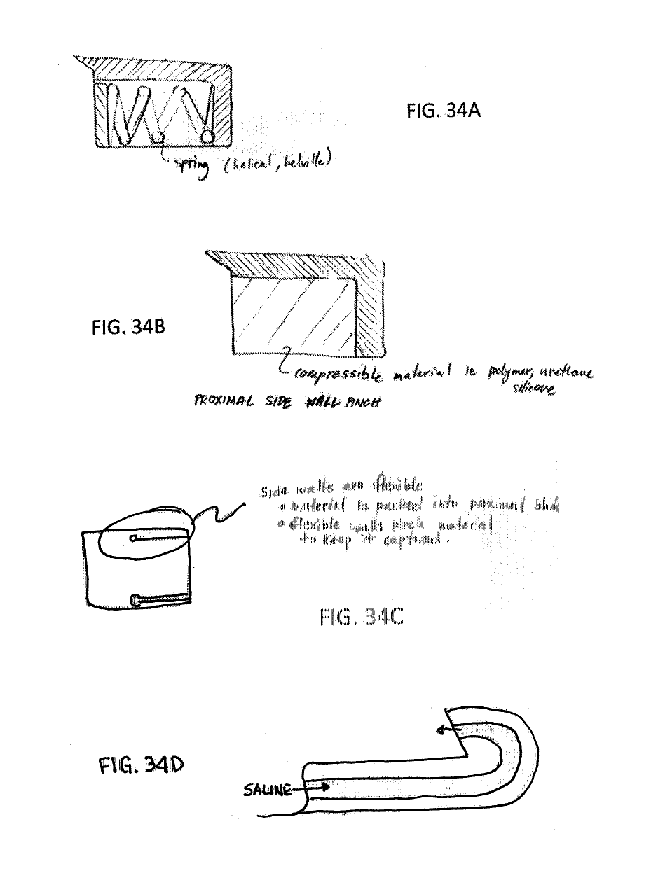

FIGS. 33A to 34D illustrate various embodiments of the tissue modification device tissue management. In some embodiments, it may be desirable to provide a tissue collection and/or tissue pass through mechanism at the distal end of the tissue modification device. As shown in FIGS. 33A and 33B, the distal blade (FIG. 33B) may have a channel or opening through which the cut tissue may pass. As shown in FIG. 33A, the tissue may pass distally through the distal blade and exit through the distal end of the device. As shown the distal end of the device may have a curved atraumatic end. Alternatively, as shown in FIG. 33C, the distal blade may include a channel and opening that guides the cut tissue up and out through the upper surface of the device. In an alternative embodiment, the proximal and/or distal blade may include a storage compartment. As shown, the compartment may be spring loaded such that the compartment will expand as it is filled with cut tissue. As shown in FIG. 34B, the compartment may be filled with a compressible material such that the compartment will expand as it is filled with cut tissue. As shown in FIG. 34C, the side walls of the distal and/or proximal blade may be flexible and/or expandable such that the compartment may expand as it is filled with cut tissue. The tissue modification device may further include a channel through which suction and/or irrigation may be run, such that the tissue may be flushed out or sucked in from distal cutting end of the tissue modification device.

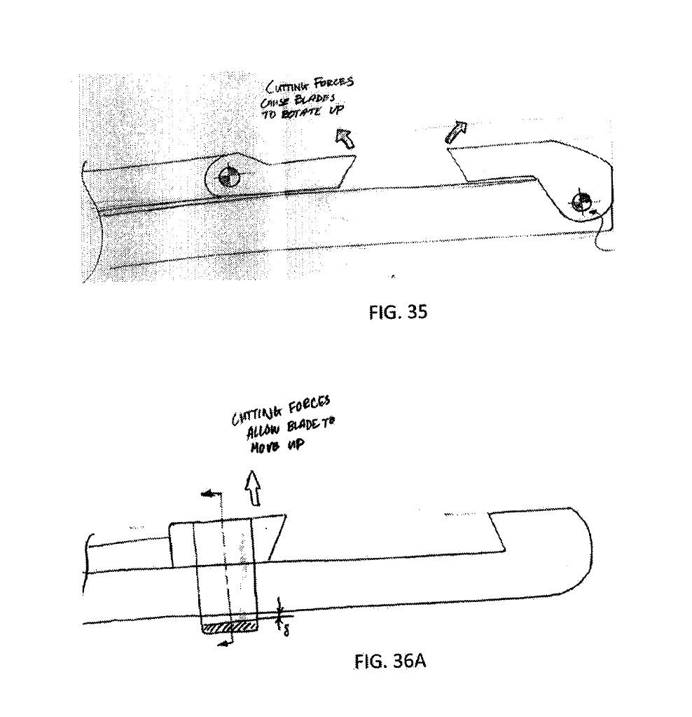

The tissue modification device as described herein will be generally used in a very narrow and/or compressed portion of the spine of the patient. Therefore, it may be desirable for the device to have a thin and/or narrow cross section. However, if the device has a thin and/or narrow cross section, it may not be able to remove as much tissue as it would with a larger cross section. Therefore, it may be desirable for the blades and/or the bit opening of the device to expand once deployed and/or as it is cutting tissue. FIGS. 35 to 37 illustrate embodiments of the tissue modification device having an expandable blade region. As shown in FIG. 35, the proximal and/or distal blade may be coupled to the tissue modification device via a rotatable/pivotable joint, such that as the blades co-act against the tissue, they rotate up and therefore cut more tissue with a single bite. Alternatively, as shown in FIGS. 36A and 36B, the proximal blade may be coupled to the tissue modification device via a strap. The strap may be expandable or otherwise allow the proximal blade to move up and cut a deeper bite of tissue. Alternatively or additionally, a strap may be coupled to the distal blade (not shown). As shown in

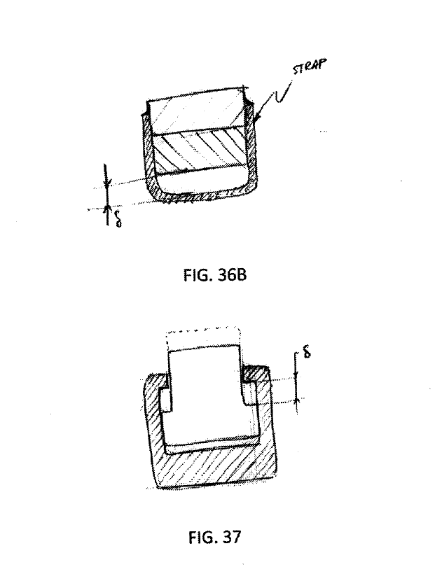

FIG. 37, the blade may ride within a channel or opening that is larger than the cross section of the blade such that the blade may move up while cutting and cut a deeper bite of tissue.

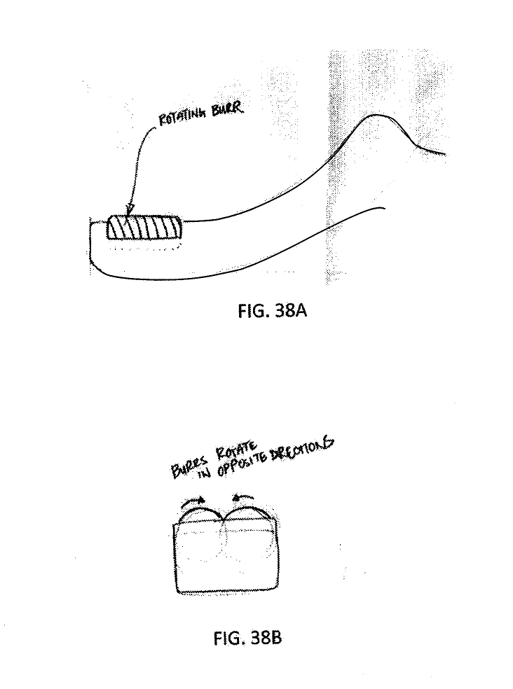

FIGS. 38A and 38B illustrate a tissue modification device having an alternative tissue modification mechanism. Any of the devices as described herein may include a powered cutting mechanism. In some embodiments, the movement of the co-acting blades may be powered. Alternatively, the blades may be replaced by a rotating burr mechanism. In some embodiments, as shown, the burrs may rotate in opposite directions.

In some embodiments, the device described herein may be disposable. In some embodiments, the device described herein may include a neural localization element. In some embodiments, the neural localization element may be at least one electrode configured to emit stimulation from at least one side (e.g. the cutting side) of the device. The stimulation element may be coupled to one of the blades and/or to the elongate body. Alternatively, it may be coupled to the distal tip of the device. In some embodiments, a threshold stimulation amount may elicit an EMG response in the patient, and depending on the magnitude of the stimulation amount, the location of the nerve with respect to the device may be determined. Alternatively, in some embodiments, the neural localization element may include a visualization element such as a camera, endoscope, or microscope. For example, the device may include at least one fiber optic bundle, CCD image sensor, or CMOS image sensor, or any combination thereof. In some embodiments the visualization element may be positioned on the distal tip of the device. Alternatively, the visualization element may be positioned such that it may visualize the window between the cutting blades. The visualization element may be coupled to one of the blades and/or to the elongate body.

As described above, the device may further include a tissue capture region or mechanism configured to capture and/or store tissue that has been cut and/or modified by the device. For example, a portion of the shaft coupling the cutting blades to the proximal handle may include a chamber that receives, collects, and stores tissue.

In some embodiments, the device may further include suction and/or irrigation capabilities. Suction and/or irrigation may aid in visualization, tissue capture, tissue modification, and/or tissue release from the device or into the storage region, bleeding management, and/or any other suitable function. In one example, the suction and/or irrigation capabilities may run from the distal tip, through the proximal handle, and include connection port(s) sized and configured to couple to standard suction and/or irrigation sources.

Any of the procedures described herein can be done in combination with other techniques including an open or minimally invasive decompression procedure where tools such as rongeurs and powered drills are used to remove tissue primarily around the proximal end of nerve root (lateral recess). Such techniques may include laminotomies, etc.

Also described herein are variations of devices (e.g., tissue modification and/or removal devices) that include a distal tissue modifying region having one or more tissue cutting elements, a connection region, connecting the tissue modifying region, and a proximal handle. The tissue cutting elements are typically movable elements that are configured to be actuated from the proximal handle, and move relative to an adjacent distal protective region. The distal protective region may act as shield.

For example, described herein are surgical instruments for cutting tissue that include a distal body attached or configured to attach to a handle assembly, and a flexible blade (or blades) that is connected or connectable to the distal body so that it can move relative to an adjacent guide or protector on the distal body. The guide or protector may provide a track or path for the blade(s).

In some variations, these devices are adapted so that the distal body is not fixed and rigid, but can be adjusted. In particular, the distal end of the device can be bent, curved, or adjusted to configure the shape and/or angle of the distal body. In some variations the distal end of the device (including any shield, guide or protector region of the distal end) can be flexible or bendable, and may be locked or secured in position once a desired configuration is achieved. In some variations the distal end of the device is configured as a guide that guides the flexible portion of the device which may include the cutting element(s) (e.g., blades). The flexible region can bend to conform to the shape of the blade guide. The guide, which may be referred to as a blade guide, may be flexible, to bend or be moved between various bent or straight configurations; the blade may then follow this end in the guide.

Any of the cutting elements described above may be used. As mentioned, in some variations, the cutting element or blade(s) is configured as a unitary blade, having a distal cutting region, a proximal portion and a medial portion between the distal and proximal regions. These different regions may be formed of different materials and/or structures that are connected or coupled together. In some variations, the blade is configured as a series of cutting cables or belts that move either continuously or in alternating direction across the distal end region of the device to cut tissue. Thus, the blade or blades do not move in a reciprocal linear manner, but may move in a rotational and/or non-reciprocating manner.

In some variations, as illustrated above, the device may be comprised of two or more regions, such as a proximal and distal region (and any intermediate regions), and these regions may be connected by joints (thus, may be comprised of flexibly connected stiff members). Different regions may be formed of different materials, and may have different functions.

The devices may also include one or more cables, rotary blades, belts, or the like, which may span these different regions. For example, in some variations, the device includes an actuator that moves one or more cutting element in a rotary manner, or a continuous (non-reciprocating) manner.

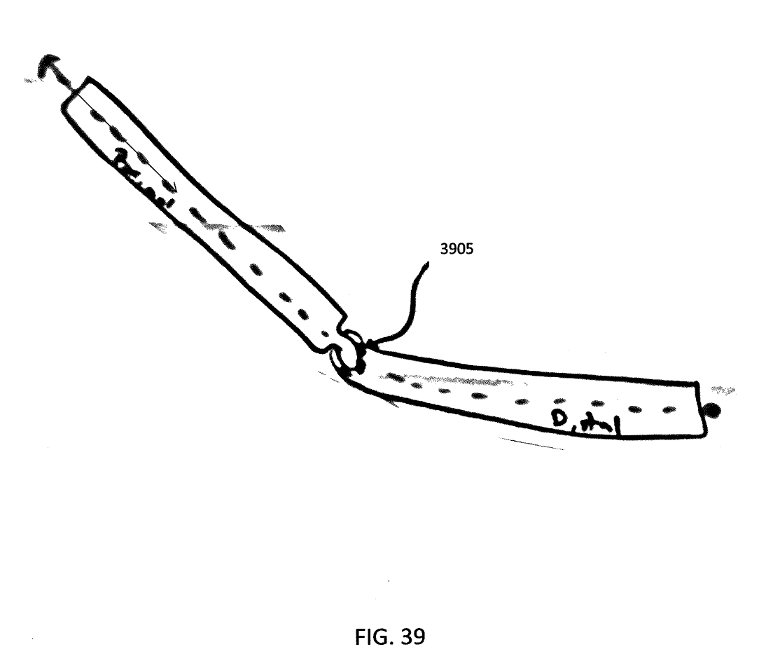

FIG. 39 shows one example of a device having a distal end region including a guide and a cutting region, where the entire distal end region (including guide) is not rigidly attached to the proximal region, but is bendable. Once bent, or between bends, the device may be locked in a position (one or more preset position or angles, or a continuously selectable position/angle). In FIG. 39, three preset positions are included, based on three spring-loaded set points that may be used to lock the bend in the distal (flexible guide) region. In some variations a control on the proximal end (A) may be used to lock the angle 3905; for example, a push/pull mechanism may lock the bending joint at the distal en once a bend angle has been chosen. As mentioned in some variations the device may include a plurality of pre-set positions for the bend angle. For example, a pin may be used to engage one or more set positions (e.g., holes, recesses, etc.) in the joint region to hold it in place. Thus, the device (the joint region) may be “indexed” to select one or more angles. A spring-loaded mechanism may be used to set and hold the distal end of the guide member in position. Once the angle/bend in the guide member has been set, in some variations the flexible cutting element may be moved against the guide member to cut tissue distally. In some variations a cutting element may extend across the joint.

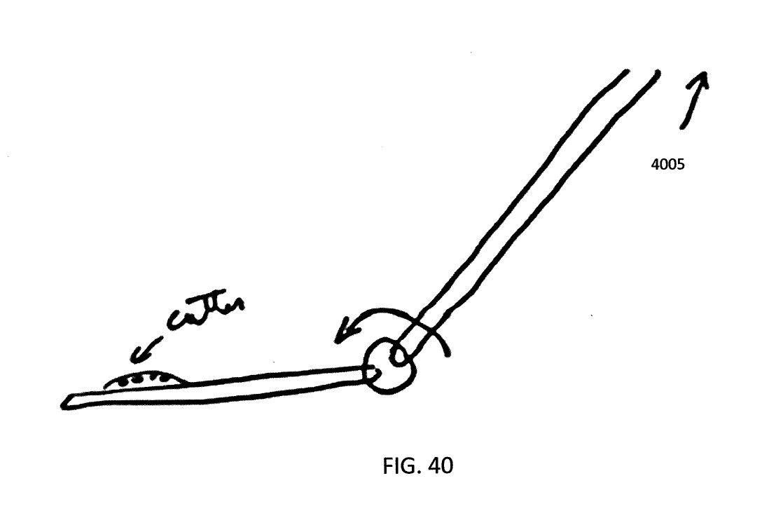

FIG. 40 shows another variation of a device having a hinged distal end region on the guide member that can be adjusted and locked into position. In this example, the angle may be adjusted and/or locked in position by controlling a screwing mechanism at the proximal end (e.g., handle region).

In some variations the device includes a flexible region (e.g., including a guide or track region) that can be locked into place using one or more pull wires/tendons to lock a chosen position. The device may be held in place by one or more tendons that are collectively or individually tightened to lock the position. For example, a cable system may be used to tighten and lock the distal end in a particular shape.

In any of the variations, the device may be configured with a plurality of pre-determined bends or shapes for the distal end (e.g., guide) region. Thus, in any variation of these surgical devices, the device may include a flexible guide (e.g., distal end region) for use with the cutting elements, but the guide can be locked into a selected position.

In some variations the device is flexible and includes lockable links/tubes that are secured by cables. Thus, the distal end of the device may be passively or actively moved and locked by pulling the cables 4005 to pull the links in place.

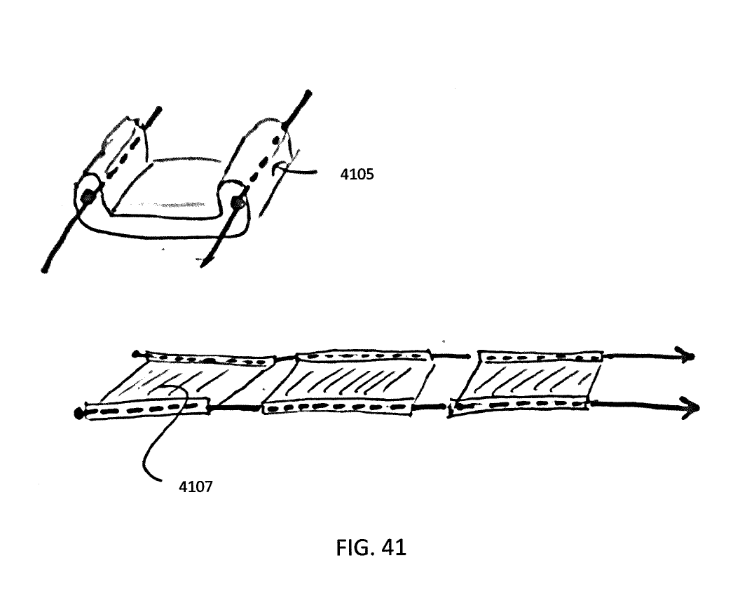

FIG. 41 shows one variation of a device having a flexible region formed of a plurality of links connected together so that they may be anchored or locked in position relative to each other to hold a position. In this example, the individual links 4105 may be rigid, but they are flexibly held together so that they can be locked to form an overall rigid structure. For example, in FIG. 3, the device can be flexible and the overall shape easily changed either by pushing/pulling on the device, or by using a controlling member such as one or more internal tendons. A selected shape can then be locked by pulling pull-wires to anchor the links 4107 together.

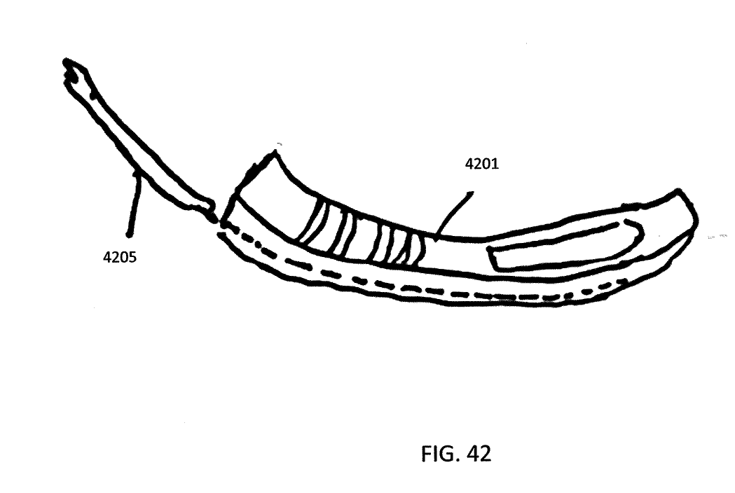

In some variations, such as the one shown in FIG. 42, the distal end region is flexible but can be made rigid by using a rigidifying member such as a rod or other material that is added to the distal end to have it conform to a bend shape configuration. Thus a stiffening member 4205 can mechanically transform the flexible distal end 4201 or guide region into a rigid region. For example, a fixed shape wedge (rod, post, wire, etc.) may be inserted into the distal end of the device to make it rigid; different fixed shapes may be selected to hold different shapes.

In some variations an overtube may be used to rigidify the flexile distal guide region. For example, a ridged cannula may be applied over the flexible distal end of the device. In both the overtube and the insertable stiffener variations the flexible device may be inserted into the body first, than a rigidifying member may be added to make it stiff within the tissue. Alternatively, the flexible distal end may be adapted for insertion by inserting a rigidifying member outside of the target body region before insertion (or re-insertion).

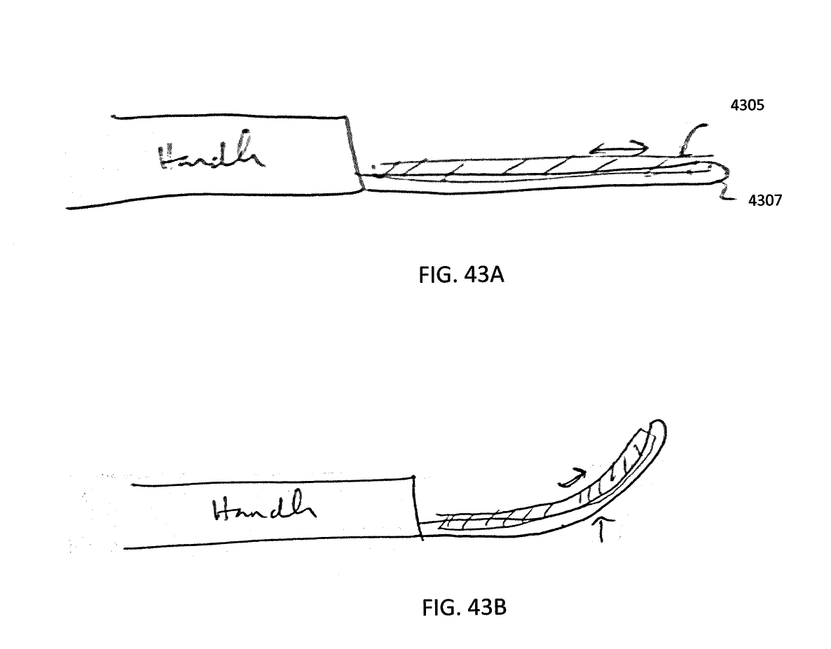

In some variations, the device may be malleable from a straight into a bent shape. For example, in one variation shown in FIGS. 43A and 43B, the device is bendable by application of force from a tool (e.g., a “bending tool” not shown) to convert the device into a bent shape.

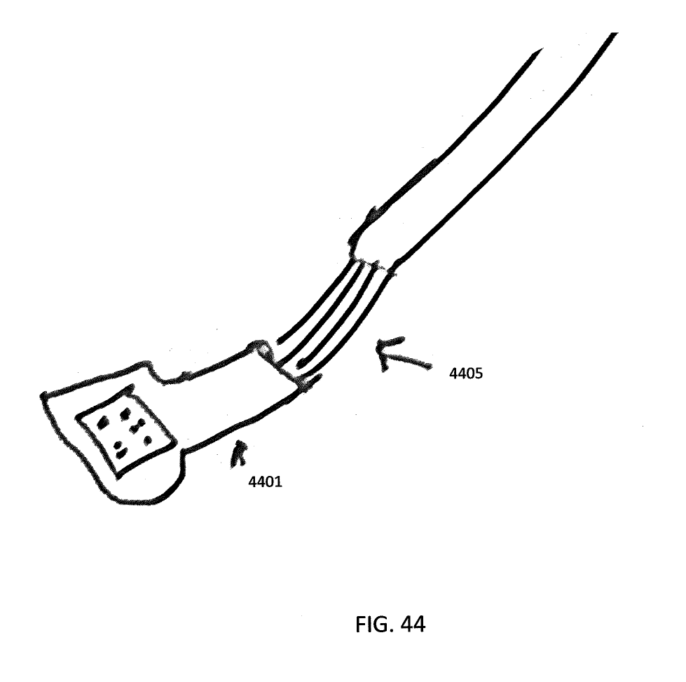

The distal end of the device 4307 and/or the cutting element(s) 4305 may comprise one or more members that extend from a proximal region of the device to a distal end region of the device and are connected by a flexible region, as shown in FIG. 44. For example, the device may be formed of two or more regions that are connected. In some variation, these regions are linked by a flexible region (e.g., flexible wires 4405). The distal end 4401 includes one or more cutting (e.g., blade) elements, for modifying the tissue, as discussed above.

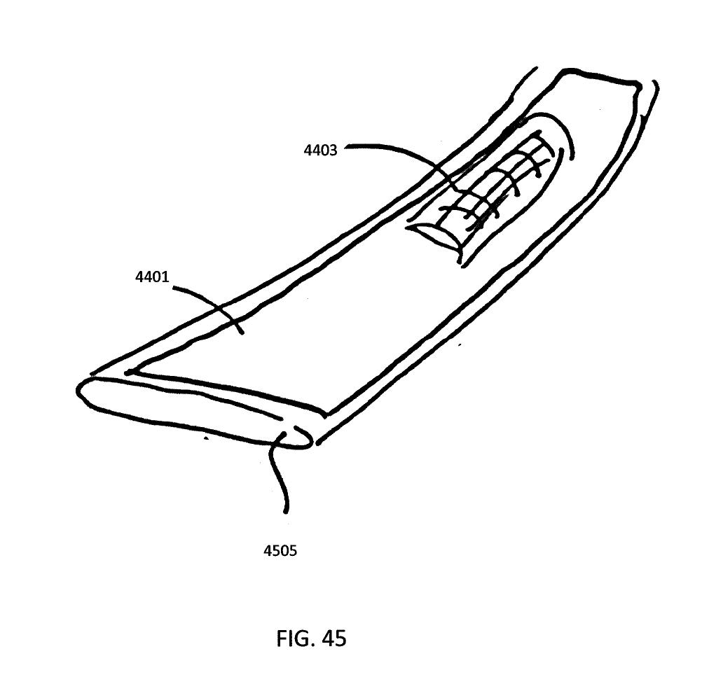

Some variation of the devices described herein include one or more anchoring members that provide leverage for the tissue modifying member to when modifying tissue, rather than relying on any stiffness of the device. For example, FIG. 45 shows the distal end of a device, a surgical tool, that includes a flexible cutting element 4503 configured to move (e.g., reciprocate) in a flexible guide member 4501; an expandable (e.g., inflatable balloon4505) region behind the cutting element and/or guide member may be used to anchor and provide support/leverage for the blade as it removes tissue. In FIG. 45, inflating the balloon may drive the blade member against the tissue to be modified and may also anchor it into position.

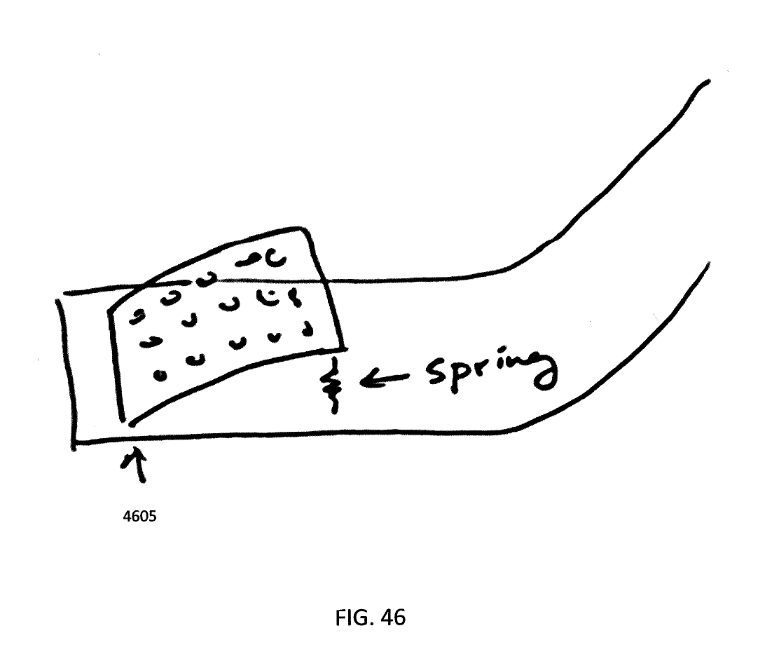

FIG. 46 shows another example of a device in which the tissue modifying region of the device (the region from which cutting members extend) are configured to extend away from the device and be driven against the target tissue; the cutting member(s) may then be moved against the target tissue to cut the tissue. In this example the cutting elements are spring loaded to drive them against the tissue and hinged 4605 on one side connecting the cutting elements to the device. A spring may be controllably released when the device is near the target region. This expansion of the region of the device near the cutting surface may be used in different/alternative configurations to anchor and brace or support the blade so that the cutting surface can apply force to cut the tissue.

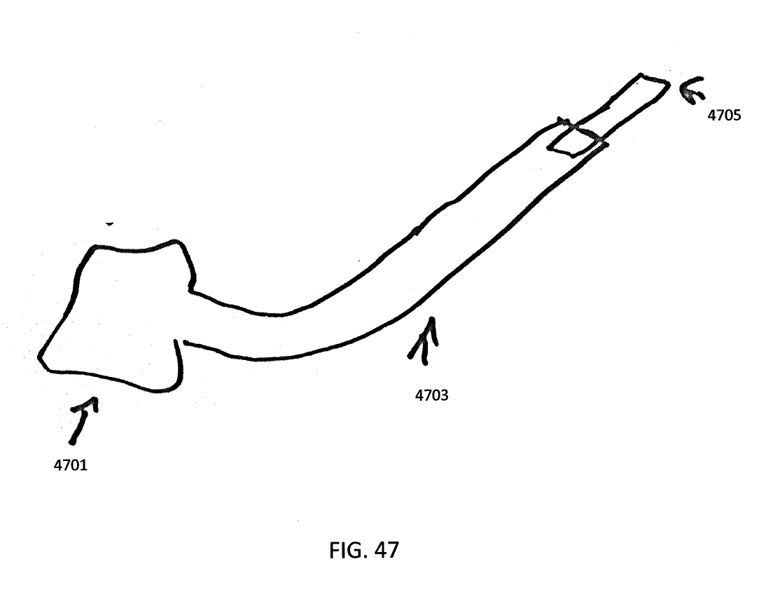

Any appropriate variation of the blade/cutting element may be used, including segmented or non-integral devices, such as those shown in FIG. 47. In this example, the distal end of the device is formed of three regions, a proximal region, a middle region and a distal region, that are each formed of a different material. The cutting window may be formed on the distal end of the device. The different regions may be joined or connected directly or by a coupling region, such as a hinge, flexible coupling region, or the like. For example, the distal 4701 region may comprise a polymeric or metal (relatively stiff) material, including the region forming the cutting window. The intermediate region 4703 may be formed of a polymeric material, and the proximal region 4705 may be formed of a metallic material (e.g., metal). These different regions my therefore has different material properties.

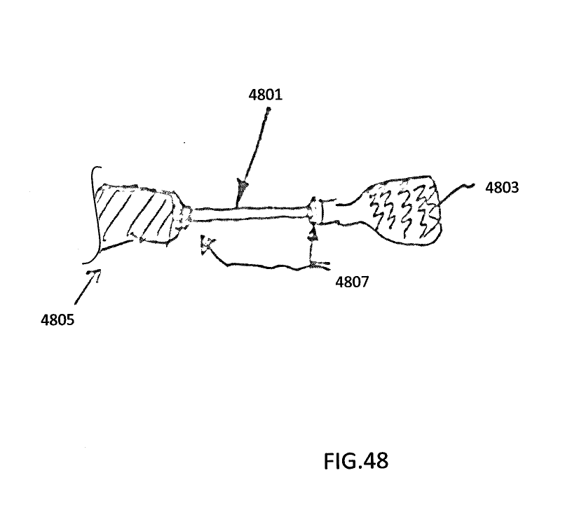

FIG. 48 shows a device (e.g., the distal end of a device) having different regions that also include a proximal 4805, middle 4801 and distal 4803 end. In this example, the distal end region includes a plurality of cutting elements and is coupled via a welded joint 4807 to a flexible wire forming the middle region. The flexible wire is coupled to a proximal end region. The proximal end region can then be itself part of (or connected) to a drive mechanism to move the distal end of the blade and thereby cut tissue. In some variations the blade is rotated; in some variations, the blade may be reciprocated, either (or both) back and forth or side to side.

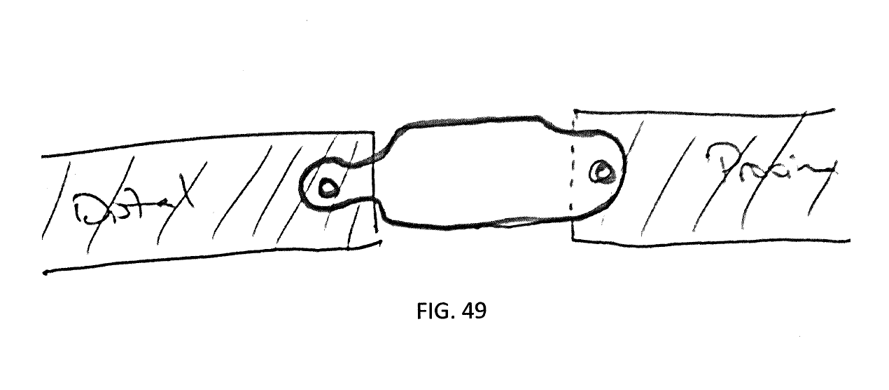

Other devices having different functional and/or compositional regions may include devices that are hinged or segmented. For example, FIG. 49 shows one variation of a device having proximal and distal region as that are hinged to a middle region. The middle region between the proximal and distal ends may have a different stiffness than the proximal and distal regions.

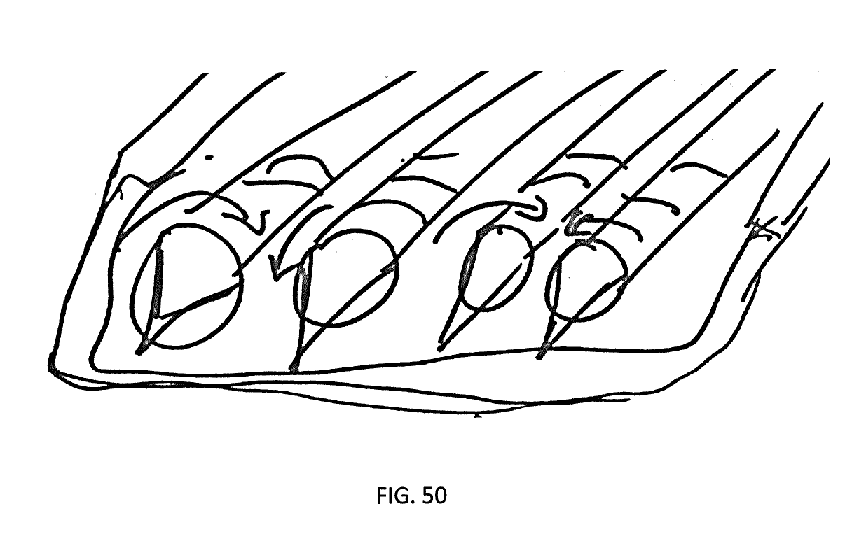

In general, the devises described herein may include a cutting element that is actuated by any appropriate type of movement. For example, the cutting elements may be configured for rotary, linear, or and/or reciprocating motion. FIG. 50 shows one variations of a device configured for a cutting element having rotary motion, in which a plurality of rotating auger elements are arranged in parallel and rotate to cut tissue on one side of the device. The rotation may be driven from the proximal end of the device using a rotational driver (e.g., in the handle). A drive shaft or multiple drive shafts may connect to the device. In some variations, the device is configured so that adjacent augers rotate in opposite directions. The auger elements may be flexible wires, shafts, tubes or the like. The surfaces of the augers may be configured to cut tissue. For example, in some variations the surfaces are threaded (e.g., screw-like) for cutting the tissue. In some variations, the devices are configured so that they also remove the tissue that is cut. For example, in the auger-type devices, the augers or adjacent regions may be hollow and configured to store and/or remove the tissue. Suction may be applied to remove the tissue.

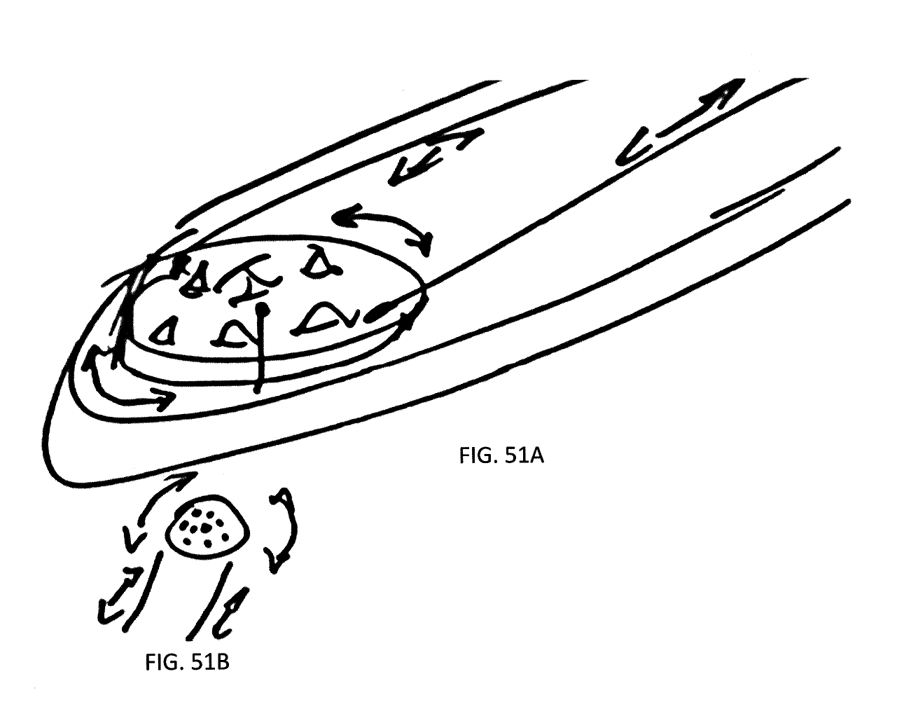

FIGS. 51A and 51B illustrates another variation of a device configured to include a rotational cutting element, shown in FIG. 51 as a disc blade that is located at the distal end of the device. In this variation, the distal end includes a blade disc that rotates (e.g., clockwise, counterclockwise, or back and forth between clockwise and counterclockwise). The rotation of the disc drives the cutting elements projecting from the disc to cut the tissue. The disc may therefore include a plurality of blade elements or projections, as shown in FIG. 51B. The rotation of the blade may be driven by a single rotating drive shaft, or by a drive-train system including a loop of material that is pulled/pushed to drive rotation of the disk. Alternatively, a pair of drive members (rods, wires, tendons, shafts, etc.) may be used. For example, as shown in FIG. 51A, a pull wire/tendon may be secured to either side of the edge region of a disc; the disc may be pinned at a rotation point (e.g., the center), and alternately pulling (and/or pushing) each wire may rotate the disc back and forth around the pinned point.

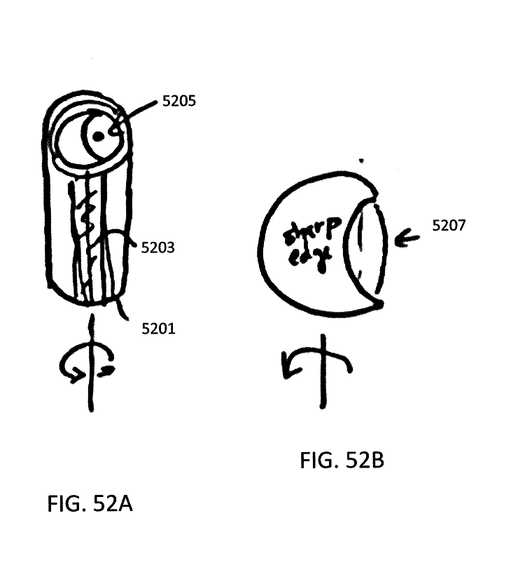

In some variations a cutting element may include a rotating cutter at or near the distal end that cuts as it rotates using a cutting element coupled to a drive shaft to rotate the cutting element. Thus, the device may include a milling cutter (e.g., a ball nose cutter, face mill cuter, or the like). The cutting element may be configured to mill the material as the cutter is rotated. FIGS. 52A and 52B illustrate one example of a cutter having a rotatable ball cutter 5205 with a scoop region cut-out 5207 that allows cutting of the tissue as the ball head is rotated by a drive shaft 5203. The drive shaft may be hollow and the hollow region 5201 may be continuous with the opening in the ball, so that tissue may be secured within the drive shaft. In some variations a plurality of such milling cutters may be included in the device.

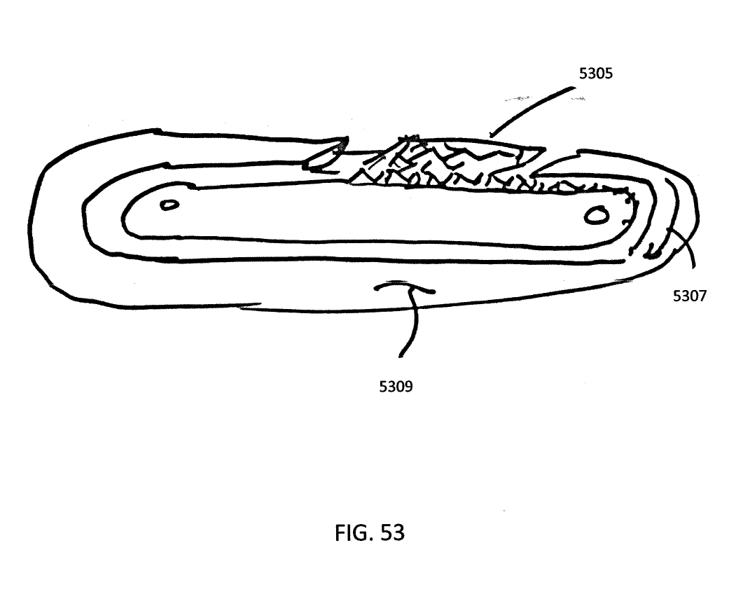

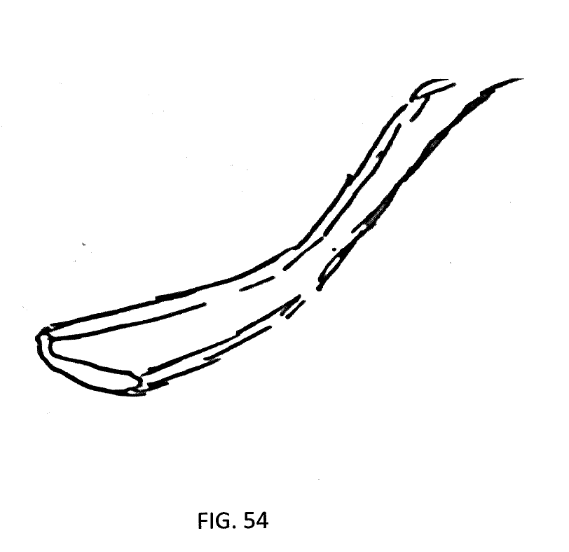

Some variations of the cutters described herein may be continuously driven by a belt or other drive element. For example, in some variations the device includes a cutting element that is driven by a rotating wire, ribbon, etc. that can be continuously or intermittently driven around the distal end region of the device for cutting tissue. FIG. 53 is a schematic showing one variation of such a device 5309, in which a belt includes or is connected to a flexible blade 5305 that rotates to cut tissue. The belt may be driven in a single direction or back and forth 5307 (e.g., clockwise, and counterclockwise). In some variations the belt runs along the length of the device, while in other variations the belt runs around the distal end region of the device, and a drive shaft may be used to drive rotation of the belt. In one variation, the cutting element comprises a plurality of cutting wires (e.g., Gigli-type wires) that have tissue cutting features for cutting tissue as the wire is driven around the device.

Many of the variations described herein include a guide region, as mentioned above. In some variations, the guide region controls the blade shape near the distal end, and may provide a surface or surfaces for the blade to move against when cutting. The guide may include a track or other region holding the blade in position. In some variations, the guide region does not include tracks or other regions holding the blade, and the blade is free to move over or against the guide region. FIG. 54 shows one variation of this device.

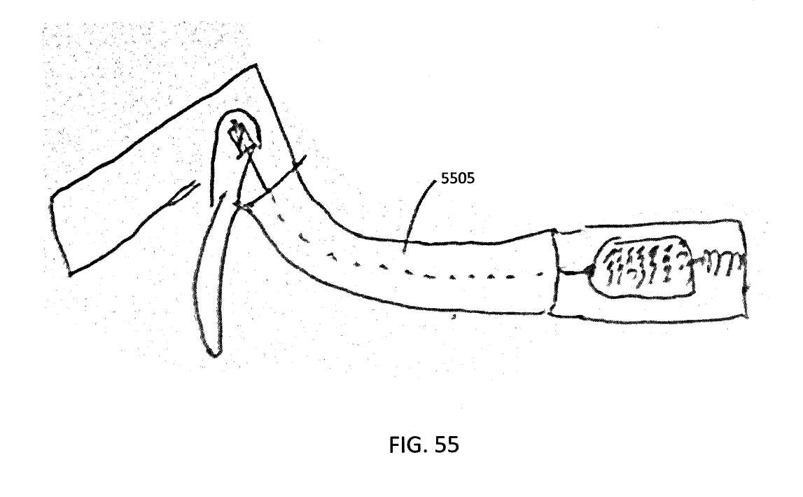

As mentioned above, any of the devices described herein may include an actuator to drive movement of the tissue modification region at the distal end. Any appropriate actuator may be used, including a motor, a drive shaft, and the like. The actuator may be manual or automatic. For example, in some variations, the actuator is a manual actuator for applying linear motion to actuator the cutting elements (e.g., blade) at the distal end. FIG. 55 illustrates one variation of such a manual actuator. In this example, the handle at the proximal end includes a squeeze trigger or grip that can be squeezed to pull a transmission element 5505 (e.g., tendon, wire, etc.) to transmit the linear motion to the distal end, where a cutting element is configured to be pulled and/or pushed by the transmission element. A spring element connected to the cutting member may provide a restoring force to help move the cutting element in opposition to the motion from squeezing the handle.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An ultra low-profile rongeur device for cutting a target tissue, the device comprising:
an elongate body having a distal portion having a height and width, wherein the distal portion of the device is configured to be passed into an epidural space and has a height that is less than about 3 mm;
a first blade movably disposed across the width of one side of the distal portion of the elongate body configured to cut target tissue;
a handle at the proximal end of the body, wherein the handle includes an actuator configured to drive the first blade towards a second blade to cut target tissue.

2. The device of claim 1, wherein the distal portion of the elongate body is curved.

3. The device of claim 2, wherein the first blade is configured to move along the curved distal portion of the elongate body.

4. The device of claim 1, wherein the actuator is configured to pull the second blade toward the first blade.

5. The device of claim 1, wherein the actuator is configured to push the first blade toward the second blade.

6. The device of claim 1, wherein the device is configured to cut target tissues within the lateral recess of a spine.

7. The device of claim 1, wherein the width is significantly greater than the height of the distal portion of the elongate body.

8. The device of claim 1, further comprising a rigid shaft region between the distal portion of the elongate body and the proximal handle.

9. The device of claim 8, wherein a distal portion of the device is curved such that there is an angle between the rigid shaft and the distal portion of the elongate body.

10. The device of claim 9, wherein the angle is between 180 degrees and 90 degrees

11. The device of claim 9, wherein the angle is less than 90 degrees.

12. The device of claim 1, wherein the distal portion has a width that is greater than about 4 mm.

13. The device of claim 1, further comprising an opening through the first or second blade through which cut tissue may pass.

14. The device of claim 1, further comprising one or more flexible tendons coupled to the first blade and the actuator and configured to move the first blade relative to the second blade.

15. The device of claim 14, wherein the one or more flexible tendons comprise a plurality of adjacently arranged wires.

16. An ultra low-profile rongeur device for cutting a target tissue, the device comprising:
an elongate body comprising a distal portion having a height and width and an elongate rigid shaft portion, wherein the distal portion of the device has a curve relative to shaft, further wherein the distal portion is configured to be passed into an epidural space and has a height that is less than about 3 mm;
a first blade that is movably disposed across the width of one side of the distal portion of the elongate body configured to cut target tissue;
one or more flexible tendons coupled to the first blade and configured to drive the first blade along the curve of the distal portion and against a second blade in the distal end region to cut target tissue.

17. The device of claim 16, further comprising a handle at the proximal end of the body, wherein the handle includes an actuator configured to move the one or more flexible tendons.

18. The device of claim 16, wherein the distal portion has a width that is greater than about 4 mm.

19. The device of claim 16, further comprising an opening through the first or second blade through which cut tissue may pass.

20. The device of claim 16, wherein the flexible tendons comprise a Nitinol member.

21. The device of claim 16, wherein the flexible tendons comprise a plurality of adjacently arranged wires.
